(19) United States
(12) Patent Application Publication
(10) Pub. No.: US 2025/0213574 A1
(43) Pub. Date: Jul. 3, 2025

(54) THERAPEUTIC COMBINATIONS COMPRISING UBIQUITIN- SPECIFIC-PROCESSING PROTEASE 1 (USP1) INHIBITORS AND CHEMOTHERAPY AGENTS

(71) Applicant: KSQ Therapeutics, Inc., Lexington, MA (US)

(72) Inventors: Andrew Alistair WYLIE, Newton, MA (US); Anne Louise CADZOW, Boston, MA (US); Pamela Jean SULLIVAN, Dedham, MA (US); Erica Rose TOBIN, Milton, MA (US)

(21) Appl. No.: 18/852,936

(22) PCT Filed: Apr. 7, 2023
(86) PCT No.: PCT/US2023/065502

§ 371 Date: Sep. 30, 2024

Related U.S. Application Data
(60) Provisional application No. 63/380,735, filed on Oct. 24, 2022.
(60) Provisional application No. 63/329,228, filed on Apr. 8, 2022.

(51) Int. Cl. A61K 31/519; A61K 31/407; A61K 31/4745; A61K 33/243; A61P 35/00

(52) U.S. Cl. CPC A61K 31/519; A61K 31/407; A61K 31/4745; A61K 33/243; A61P 35/00

(57) ABSTRACT

The present disclosure provides combinations comprising (i) a chemotherapy agent and (ii) a ubiquitin-specific-processing protease 1 (USP1) inhibitor having Formula I: (I), or a pharmaceutically acceptable salt or solvate thereof. The present disclosure is also directed to methods of treating cancers, delaying, reducing, and/or preventing rebounding of a tumor comprising administering the combinations are provided.

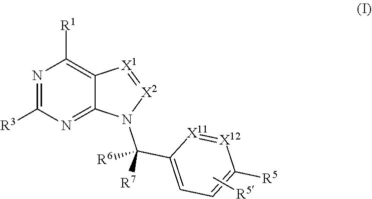

### CROSS REFERENCE TO RELATED APPLICATIONS

This application claims the benefit of U.S. Provisional Application Nos. 63/380,735, filed Oct. 24, 2022 and 63/329,228, filed Apr. 8, 2022, each of which is hereby incorporated by reference in its entirety.

### REFERENCE TO A SEQUENCE LISTING SUBMITTED ELECTRONICALLY VIA EFS-WEB

The content of the electronically submitted sequence listing (Name 4195_025PC03_Seglisting_ST26.txt; Size: 2,997 bytes; and Date of Creation: Apr. 6, 2023) is incorporated herein by reference in its entirety.

### BACKGROUND OF THE DISCLOSURE

### Field

The present disclosure provides combinations of chemotherapy agents and ubiquitin-specific-processing protease 1 (USP1) inhibitors. Methods of treating cancers, delaying, reducing, and/or preventing rebounding of a tumor comprising administering the combinations are provided.

### Background

Ubiquitin is a small (76 amino acid) protein that is post-transcriptionally attached to target proteins. The consequence of ubiquitination is determined by the number and linkage topology of ubiquitin molecules conjugated to the target protein. For example, proteins exhibiting lysine 48-linked poly-ubiquitin chains are generally targeted to the proteasome for degradation, while mono-ubiquitination or poly-ubiquitin chains linked through other lysines regulate non-proteolytic functions, such as cell cycle regulation, DNA damage repair, transcription, and endocytosis. Ubiquitination is a reversible process, and enzymes called deubiquitinases remove ubiquitin from target proteins.

USP1 is a deubiquitinase that plays a role in DNA damage repair. USP1 interacts with UAF1 (USP1-associated factor 1) to form a complex that is required for the deubiquitinase activity. The USP1/UAF1 complex deubiquitinates mono-ubiquitinated PCNA (proliferating cell nuclear antigen) and mono-ubiquitinated FANCD2 (Fanconi anemia group complementation group D2), which are proteins that play important functions in translesion synthesis (TLS) and the Fanconi anemia (FA) pathway, respectively. The USP1/UAF1 complex also deubiquitinates Fanconi anemia complementation group I (FANCI). These two pathways are essential for repair of DNA damage induced by DNA cross-linking agents, such as cisplatin and mitomycin C (MMC).

Chemotherapy agents induce a variety of different DNA lesions which the cell must recognize and counter in order to survive. Chemotherapy agents can be divided into groups based on their mechanism of action and type of damage induced though there is considerable crossover between classes. Alkylating agents directly modify DNA and often induce bulky DNA damage that is repaired via the nucleotide excision repair pathway (NER). Platinum-based agents also induce bulky DNA damage repaired by the NER pathway and are effective in treating a wide array of cancers. A large class of agents target DNA metabolism and include DNA intercalating agents, topoisomerase poisons and antimetabolites. Many chemotherapeutic agents cause damage to DNA resulting in DNA adducts, strand breaks or stalled/collapsed DNA replication forks, the repair or restart of which often requires homologous recombination (HR) and Fanconi anemia proteins.

There remains an unmet medical need for more effective therapies, e.g., combination therapies, for the treatment of cancers.

### BRIEF SUMMARY OF THE DISCLOSURE

Combinations of (i) a ubiquitin-specific-processing protease 1 (USP1) inhibitor, and (ii) a chemotherapy agent are provided herein. Also provided herein are methods of treating a subject with cancer using such a combination.

In one aspect, the present disclosure relates to a combination comprising (i) a chemotherapy agent and (ii) a ubiquitin-specific-processing protease 1 (USP1) inhibitor, wherein the USP1 inhibitor is a compound having Formula I:

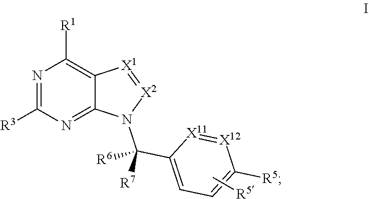

or a pharmaceutically acceptable salt or solvate thereof, wherein:
each of X¹ and X² is independently selected from N and CR²;
each of R¹ and R² is independently selected from hydrogen, halo, cyano, optionally substituted alkyl, optionally substituted alkenyl, and optionally substituted alkynyl;
R³ is an optionally substituted phenyl, optionally substituted pyridyl, optionally substituted pyrimidinyl, optionally substituted pyrazinyl, optionally substituted pyridazinyl, or optionally substituted pyrazolyl;
each of X¹¹ and X¹² is independently selected from N and CH;
R^{5′} is selected from hydrogen, optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted —O-heterocyclo, optionally substituted —O-heterocyclo-(C₁-C₂) alkyl;
R⁵ is selected from optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted —O-heterocyclo, optionally substituted —O-heterocyclo-(C₁-C₂) alkyl; or
one of R⁵ and one of R^{5′} on adjacent atoms are taken together with the atoms to which they are attached to form an optionally substituted (C₆-C₁₄) aryl ring; or one of R⁵ and one of R^{5′} on adjacent atoms are taken together with the atoms to which they are attached to form an optionally substituted heteroaryl ring; or one of R⁵ and one of R^{5′} on adjacent atoms are taken together with the atoms to which they are attached to form an optionally substituted (C₃-C₈) cycloalkyl ring; or one of R⁵ and one of R^{5′} on adjacent atoms are taken together with the atoms to which they are attached to form an optionally substituted heterocycloalkyl ring; or one of R⁵ and one of R^{5′} on adjacent atoms on the same atom to which they are attached are taken together to form an optionally substituted spirocycloalkyl ring; or one of R⁵ and one of R^{5′} on adjacent atoms on the same atom to which they are attached are taken together to form an optionally substituted spiroheterocycloalkyl ring;
each of R⁶ and R⁷ is independently selected from hydrogen, halo, cyano, optionally substituted alkyl, optionally substituted alkenyl, and optionally substituted alkynyl;
R²³ is selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, optionally substituted heterocyclo, optionally substituted heteroaryl, optionally substituted aryl, and optionally substituted cycloalkyl;
R^{31a} and R^{31b} are each independently selected from the group consisting of hydrogen, optionally substituted alkyl, optionally substituted alkenyl, optionally substituted alkynyl, hydroxyalkyl, (amino)alkyl, (alkylamino)alkyl, (dialkylamino)alkyl, alkoxyalkyl, cycloalkyl, (cycloalkyl)alkyl, (heterocyclo)alkyl, aralkyl, and (heteroaryl)alkyl; and
each of R²⁴, R²⁵, R²⁷, R^{32a}, and R^{32b} is independently selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, alkoxyalkyl, optionally substituted heterocyclo, optionally substituted heteroaryl, optionally substituted aryl, and optionally substituted cycloalkyl.

In one aspect, the chemotherapy agent is a platin, a monofunctional alkylator, a bifunctional alkylator, an antimetabolite, a topoisomerase inhibitor, or a combination thereof.

In another aspect, the chemotherapy agent is a platin selected from the group consisting of cisplatin, carboplatin, oxaliplatin, satraplatin, picoplatin, nedaplatin, and triplatin. In another aspect, the chemotherapy agent is cisplatin or carboplatin.

In another aspect, the chemotherapy agent is a monofunctional alkylator selected from the group consisting of an alkylsulfonate, a nitrosourea, and temozolomide; or a bifunctional alkylator selected from the group consisting of nitrogen mustard and mitomycin C.

In another aspect, the chemotherapy agent is an antimetabolite selected from the group consisting of gemcitabine, 5-fluorouracil, a thiopurine, and a folate analog.

In another aspect, the chemotherapy agent is a topoisomerase inhibitor selected from the group consisting of a campothecin, an anthracycline, and an etoposide.

In another aspect, the chemotherapy agent comprises a combination of leucovorin calcium, 5-fluorouracil, and oxaliplatin.

In one aspect, the present disclosure relates to a method of treating cancer in a subject comprising administering such combinations described in the prior aspects.

In another aspect, the present disclosure relates to a method of delaying, reducing, and/or preventing rebounding of a tumor in a subject comprising administering such combinations described in the prior aspects.

In one aspect, the present disclosure relates to the use of the combinations for the manufacture of a medicament for treatment of cancer.

In another aspect, the present disclosure relates to a pharmaceutical composition comprising the combinations and a pharmaceutically acceptable carrier.

In one aspect, the pharmaceutical composition is for use in the treatment of cancer.

In one aspect, the present disclosure relates to a kit comprising the combination or the pharmaceutical composition, and instructions for administering the combination to a subject having cancer.

In one aspect, the USP1 inhibitor has one of the following structures:

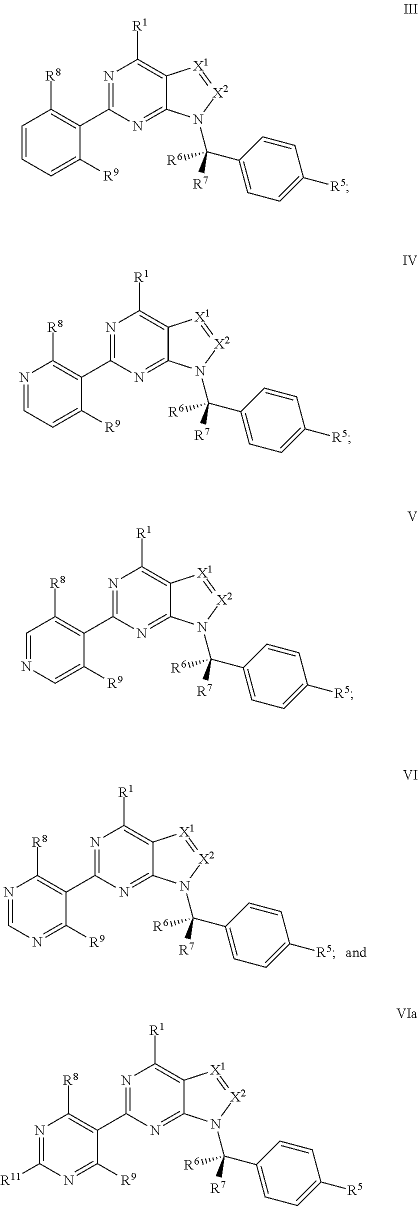

or a pharmaceutically acceptable salt or solvate thereof, wherein
each of X¹ and X² is independently selected from N and CR²;
R¹ is selected from hydrogen, halo, cyano, optionally substituted alkyl, optionally substituted alkenyl, and optionally substituted alkynyl;
R⁵ is:

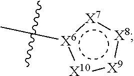

X⁶ is selected from NR¹³ and CR⁸;
X⁷ is selected from NR¹⁴ and CR¹⁹;
X⁸ is selected from NR¹⁵ and CR²⁰;
X⁹ is selected from NR¹⁶ and CR²¹;
X¹⁰ is selected from NR¹⁷ and CR²²;
each of R¹³, R¹⁴, R⁵, R¹⁶, and R¹⁷ is absent, or independently selected from hydrogen, halo, methyl, ethyl, isopropyl, cyclopropyl, methoxy, triazolyl, cyano, optionally substituted alkyl, amino, alkylamino, dialkylamino, difluoromethyl, trifluoromethyl, methylsulfonyl, and methylazetidinyl; and
each of R¹⁸, R¹⁹, R²⁰, R²¹, and R²² is independently selected from hydrogen, halo, methyl, ethyl, isopropyl, cyclopropyl, methoxy, triazolyl, cyano, optionally substituted alkyl, amino, alkylamino, dialkylamino, difluoromethyl, trifluoromethyl, methylsulfonyl, and methylazetidinyl;
each of R⁶ and R⁷ is independently selected from hydrogen, halo, cyano, optionally substituted alkyl, optionally substituted alkenyl, and optionally substituted alkynyl; and
each of R⁸, R⁹ and R¹¹ is independently selected from the group consisting of hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, or (heteroaryl)alkyl.

In another aspect, the USP1 inhibitor is a compound selected from the group consisting of:
(a) Compound 1:

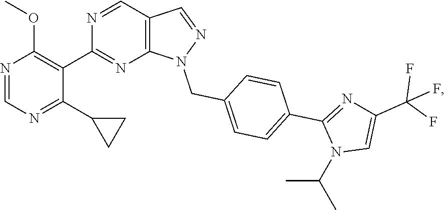 [SMILES: COc1ncnc(C2CC2)c1-c1ncc2cnn(Cc3ccc(-c4nc(C(F)(F)F)cn4C(C)C)cc3)c2n1]

(b) Compound 2:

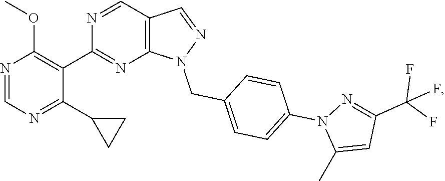 [SMILES: COc1ncnc(C2CC2)c1-c1ncc2cnn(Cc3ccc(-n4nc(C(F)(F)F)cc4C)cc3)c2n1]

(c) Compound 3:

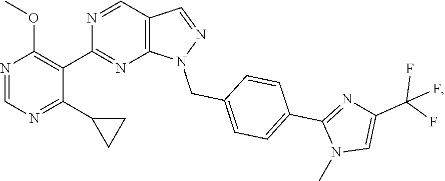 [SMILES: COc1ncnc(C2CC2)c1-c1ncc2cnn(Cc3ccc(-c4nc(C(F)(F)F)cn4C)cc3)c2n1]

and a pharmaceutically acceptable salt, hydrate, solvate, amorphous solid, or polymorph thereof. In another aspect, Compound 1, Compound 2, or Compound 3 is provided as a cocrystal. In other aspects, Compound 1, Compound 2, or Compound 3 is provided as a cocrystal with a pharmaceutically acceptable acid.

In one aspect, the USP1 inhibitor is Compound 1, or a pharmaceutically acceptable salt, hydrate, solvate, amorphous solid, or polymorph thereof. In another aspect, Compound 1 is provided as a cocrystal with a pharmaceutically acceptable acid. In one aspect, the USP1 inhibitor is a cocrystal of Compound 1 and gentisic acid, that is a cocrystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

In another aspect, the USP1 inhibitor is Compound 2, or a pharmaceutically acceptable salt, hydrate, solvate, amorphous solid, or polymorph thereof. In another aspect, Compound 2 is provided as a cocrystal with a pharmaceutically acceptable acid.

In another aspect, the USP1 inhibitor is Compound 3, or a pharmaceutically acceptable salt, hydrate, solvate, amorphous solid, or polymorph thereof. In another aspect, Compound 3 is provided as a cocrystal with a pharmaceutically acceptable acid.

In another aspect, the USP1 inhibitor is a gentisate salt of 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine.

In one aspect, the subject is a mammal. In another aspect, the mammal is a human.

In one aspect, the cancer cells of said subject are homologous recombination repair (HRR) positive.

In one aspect, the cancer or tumor is an advanced solid tumor. In one aspect, the cancer or tumor is selected from the group consisting of lung cancer, non-small cell lung cancer (NSCLC), colon cancer, bladder cancer, osteosarcoma, ovarian cancer, skin cancer, uterine cancer, peritoneal cancer, endometrial cancer, pancreatic cancer, gastric cancer, cholangiocarcinoma, and breast cancer.

In one aspect, the chemotherapy agent and the USP1 inhibitor are administered sequentially. In another aspect, the chemotherapy agent and the USP1 inhibitor are administered simultaneously.

In one aspect, the USP1 inhibitor and the chemotherapy agent are provided in separate dosage forms. In another aspect, the USP1 inhibitor is provided in an oral dosage form. In another aspect, the chemotherapy agent is provided in an injectable dosage form. In another aspect, the chemotherapy agent is provided in an oral dosage form.

In one aspect, the chemotherapy agent and the USP1 inhibitor are administered in a therapeutically effective amount sufficient to produce one or more therapeutic effects selected from the group consisting of (i) reduction in tumor size, (ii) increase in tumor regression rate, and (iii) reduction or inhibition of tumor growth.

In another aspect, the chemotherapy agent and the USP1 inhibitor delay, reduce, and/or prevent rebounding of a tumor.

In one aspect, the USP1 inhibitor, gemcitabine and carboplatin are administered. In another aspect, the subject has ovarian cancer, triple negative breast cancer, or non-small cell lung cancer.

In one aspect, the USP1 inhibitor, leucovorin calcium, 5-fluorouracil, and oxaliplatin are administered. In another aspect, the subject has colorectal cancer, pancreatic cancer, cholangiocarcinoma, esophageal cancer, or gastric cancer.

In one aspect, the cancer or tumor was previously treated with a platinum therapy. In some aspects, the cancer or tumor is platinum resistant or platinum refractory. In one aspects, the platinum therapy is selected from the group consisting of cisplatin, carboplatin, oxaliplatin, and combinations thereof.

In one aspect, the chemotherapy agent and the USP1 inhibitor are administered in an amount effective to reduce unacceptable toxicity and/or unacceptable adverse reactions of a chemotherapy agent administered as a monotherapy.

In one aspect, the present disclosure relates to the combination for use in the treatment of cancer. In another aspect, the present disclosure relates to use of such a combination for the manufacture of a medicament for treatment of cancer. In one aspect, the cancer is an advanced solid tumor. In one aspect, the cancer is selected from the group consisting of lung cancer, non-small cell lung cancer (NSCLC), colon cancer, bladder cancer, osteosarcoma, ovarian cancer, skin cancer, uterine cancer, peritoneal cancer, endometrial cancer, pancreatic cancer, gastric cancer, cholangiocarcinoma, and breast cancer. In one aspect, the cancer was previously treated with a platinum therapy. In one aspect, the cancer is platinum resistant or platinum refractory. In one aspect, the platinum therapy is selected from the group consisting of cisplatin, carboplatin, oxaliplatin, and combinations thereof.

Additional aspects and advantages of the present disclosure will be set forth, in part, in the description that follows, and will flow from the description, or can be learned by practice of the present disclosure. The aspects and advantages of the present disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE FIGURES

FIG. 1 (A) shows the tolerability of 4 mg/kg cisplatin in combination with Compound (1)-gentisic acid cocrystal in non-tumor bearing female Balb/c nude mice.

FIG. 1 (B) shows the survival of 4 mg/kg cisplatin in combination with Compound (1)-gentisic acid cocrystal in non-tumor bearing female Balb/c nude mice.

FIG. 2 (A) shows the tolerability of 5 mg/kg cisplatin in combination with Compound (1)-gentisic acid cocrystal in non-tumor bearing female Balb/c nude mice.

FIG. 2 (B) shows the survival of 5 mg/kg cisplatin in combination with Compound (1)-gentisic acid cocrystal in non-tumor bearing female Balb/c nude mice.

FIG. 3 (A) shows the tolerability of 6 mg/kg cisplatin in combination with Compound (1)-gentisic acid cocrystal in non-tumor bearing female Balb/c nude mice.

FIG. 3 (B) shows the survival of 6 mg/kg cisplatin in combination with Compound (1)-gentisic acid cocrystal in non-tumor bearing female Balb/c nude mice.

FIG. 4 shows an XRPD pattern corresponding to crystalline Form 2.

FIG. 5 shows a DSC and TGA thermogram corresponding to crystalline Form 2.

FIG. 6 (A) shows the tumor volume change of 2 mg/kg cisplatin in combination with 100 mg/kg Compound (1)-gentisic acid cocrystal in MDA-MB-436 tumor bearing female NOD SCID mice.

FIG. 6 (B) shows the tolerability of 2 mg/kg cisplatin in combination with 100 mg/kg Compound (1)-gentisic acid cocrystal in MDA-MB-436 tumor bearing female NOD SCID mice.

FIG. 7 (A) shows the tumor volume change of 5 mg/kg cisplatin in combination with 100 mg/kg Compound (1)-gentisic acid cocrystal in HBCx-8 tumor bearing female Athymic nude mice.

FIG. 7 (B) shows the tolerability of 5 mg/kg cisplatin in combination with 100 mg/kg Compound (1)-gentisic acid cocrystal in HBCx-8 tumor bearing female Athymic nude mice.

FIG. 8 (A) shows the tumor volume change of 3 mg/kg cisplatin in combination with 100 mg/kg Compound (1)-gentisic acid cocrystal in HBCx-8 tumor bearing female Athymic nude mice.

FIG. 8 (B) shows the tolerability of 3 mg/kg cisplatin in combination with 100 mg/kg Compound (1)-gentisic acid cocrystal in HBCx-8 tumor bearing female Athymic nude mice.

### DETAILED DESCRIPTION OF THE DISCLOSURE

One aspect of the present disclosure is based on the use of a combination of (i) a chemotherapy agent and (ii) a ubiquitin-specific-processing protease 1 (USP1) inhibitor. The combinations are useful for inhibiting a USP1 protein and for treating diseases, disorders, or conditions, e.g., cancer, that are responsive to a chemotherapy agent and/or inhibition of a USP1 protein.

In some aspects, the combination of a USP1 inhibitor and a chemotherapy agent provide a synergistic effect.

In some aspects, the USP1 inhibitor and the chemotherapy agent are in therapeutically effective amounts sufficient to produce a therapeutic effect comprising: (i) a reduction in size of a tumor, (ii) an increase in cancer tumor regression rate, (iii) a reduction or inhibition of cancer tumor growth, and/or (iv) a reduction of the toxicity effects of a chemotherapy agent administered as a monotherapy. In some aspects, the USP1 inhibitor and the chemotherapy agent can delay, reduce, or prevent rebounding (rapid re-growth) of a tumor.

### Definitions

All technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

It is understood that embodiments described herein include “consisting” and/or “consisting essentially of” embodiments. As used herein, the singular form “a,” “an,” and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about,” as used herein, includes the recited number ±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In the context of cancer, the term “treating” includes, but is not limited to, inhibiting growth of cancer cells, inhibiting replication of cancer cells, lessening of overall tumor burden, and delaying, halting, or slowing tumor growth, progression, or metastasis.

As used herein, “delaying” means to defer, hinder, slow, retard, stabilize, suppress and/or postpone development or progression of the disease (such as cancer). This delay can be of varying lengths of time, depending on the history of the disease and/or individual being treated.

A “therapeutically effective amount” of a substance can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations. A therapeutically effective amount refers to an amount effective, at dosages and for periods of time necessary, to achieve the desired therapeutic effect.

The terms “combination,” “therapeutic combination,” “combination composition,” “combination therapy” or “pharmaceutical combination”, as used herein, can include a fixed combination in one dosage unit form, separate dosage units or a kit of parts or instructions for the combined administration where the USP1 inhibitor and the chemotherapy agent can be administered independently at the same time or separately within time intervals. A combined pharmaceutical composition can be adapted for simultaneous, separate, or sequential administration.

The combination therapy can provide “synergy” and prove “synergistic,” i.e., the effect achieved when the active ingredients used together is greater than the sum of the effects that results from using the compounds separately. A synergistic effect can include a significantly reduced effective dose for the combination of the two active ingredients as compared to the effective dose of each active ingredient when administered separately. A synergistic effect can also include a reduction in toxicity for the combination of the two active ingredients as compared to the toxicity of each active ingredient when administered separately. A synergistic effect can also be an effect that cannot be achieved by administration of any of the active ingredients as single agents. The synergistic effect can include, but is not limited to, an effect of treating cancer by reducing tumor size, inhibiting tumor growth, or increasing survival of the subject. The synergistic effect can also include reducing cancer cell viability, inducing cancer cell death, and inhibiting or delaying cancer cell growth. A synergistic effect can be attained, for example, when the active ingredients are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the compounds are administered or delivered sequentially.

A determination of a synergistic interaction between a USP1 inhibitor and a chemotherapy agent can be based on the results obtained from the assays described herein. For example, combination effects can be evaluated using the Bliss independence model. Bliss scores quantify degree of potentiation from single agents, and a Bliss score >0 suggests greater than simple additivity. In some aspects, a Bliss score greater than 10 indicates strong synergy. In other aspects, a score of 6 or greater indicates synergy. In some aspects, the Bliss score is about 6, about 7, about 8, about 9, about 10, about 11, about 12, about 13, about 14, about 15, about 16, about 17, about 18, about 19, about 20 or about 25.

As used herein, a “homologous recombination deficiency score” or “HRD score” means an algorithmic assessment of three measures of tumor genomic instability, i.e., loss of heterozygosity, telomeric allelic imbalance and large-scale state transitions.

The term “HRR+” refers to homologous recombination repair positive. HRR+ status of cancer cells can be determined based upon a biopsy. Homologous recombination repair is an important cellular mechanism by which double strand DNA breaks (DSB) are corrected with high fidelity. For purposes of the present disclosure, HRR+, HRP, HRR functional cells and HRR positive cells are used interchangeably.

The terms “administer,” “administering,” “administration,” and the like refer to methods that can be used to enable delivery of the therapeutic agent to the desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. Administration of two or more therapeutic agents includes simultaneous (concurrent) and consecutive administration in any order.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “pharmaceutically acceptable carrier,” “pharmaceutically acceptable excipient,” “physiologically acceptable carrier,” or “physiologically acceptable excipient” refer to a pharmaceutically-acceptable material, composition, or vehicle, such as a liquid or solid filler, diluent, excipient, solvent, or encapsulating material. A pharmaceutically acceptable carrier is non-toxic to recipients at the dosages and concentrations employed and is compatible with other ingredients of the formulation. The pharmaceutically acceptable carrier is appropriate for the formulation employed. In some aspects, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004 (incorporated herein by reference).

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” can be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “solvate” refers to a compound provided herein or a salt thereof, which further includes a stoichiometric or non-stoichiometric amount of solvent bound by non-covalent intermolecular forces. Where the solvent is water, the solvate is a hydrate. Where the solvent includes ethanol, the compound can be an ethanol solvate.

The term “anhydrate” as applied to a compound refers to a solid state wherein the compound contains no structural water within the crystal lattice.

A “sterile” formulation is aseptic or essentially free from living microorganisms and their spores.

The term “container” means any receptacle and closure therefore suitable for storing, shipping, dispensing, and/or handling a pharmaceutical product.

The term “insert” or “package insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

The term “disease” or “condition” or “disorder” as used herein refers to a condition where treatment is needed and/or desired and denotes disturbances and/or anomalies that as a rule are regarded as being pathological conditions or functions, and that can manifest themselves in the form of particular signs, symptoms, and/or malfunctions. As demonstrated below, combinations of the USP1 inhibitors and chemotherapy agents of the present disclosure can be used in treating diseases and conditions, such as proliferative diseases, wherein a chemotherapy agent and/or inhibition of USP1 proteins provides a benefit.

“USP1” and “ubiquitin-specific-processing protease 1” as used herein refer to any native polypeptide or USP1-encoding polynucleotide. The term “USP1” encompasses “full-length,” unprocessed USP1 polypeptide as well as any forms of USP1 that result from processing within the cell (e.g., removal of the signal peptide). The term also encompasses naturally occurring variants of USP1, e.g., those encoded by splice variants and allelic variants. The USP1 polypeptides described herein can be isolated from a variety of sources, such as from human tissue types or from another source, or prepared by recombinant or synthetic methods. Human USP1 sequences are known and include, for example, the sequences publicly available as UniProt No. 094782 (including isoforms). As used herein, the term “human USP1 protein” refers to USP1 protein comprising the amino acid sequence as set forth in SEQ ID NO:1:

(SEQ ID NO: 1)
MPGVIPSESNGLSRGSPSKKNRLSLKFFQKKETKRALDFTDSQENEEKA
SEYRASEIDQVVPAAQSSPINCEKRENLLPFVGLNNLGNTCYLNSILQV
LYFCPGFKSGVKHLFNIISRKKEALKDEANQKDKGNCKEDSLASYELIC
SLQSLIISVEQLQASFLLNPEKYTDELATQPRRLLNTLRELNPMYEGYL
QHDAQEVLQCILGNIQETCQLLKKEEVKNVAELPTKVEEIPHPKEEMNG
INSIEMDSMRHSEDFKEKLPKGNGKRKSDTEFGNMKKKVKLSKEHQSLE
ENQRQTRSKRKATSDTLESPPKIIPKYISENESPRPSQKKSRVKINWLK
SATKQPSILSKFCSLGKITTNQGVKGQSKENECDPEEDLGKCESDNTTN
GCGLESPGNTVTPVNVNEVKPINKGEEQIGFELVEKLFQGQLVLRTRCL
ECESLTERREDFQDISVPVQEDELSKVEESSEISPEPKTEMKTLRWAIS
QFASVERIVGEDKYFCENCHHYTEAERSLLFDKMPEVITIHLKCFAASG
LEFDCYGGGLSKINTPLLTPLKLSLEEWSTKPTNDSYGLFAVVMHSGIT
ISSGHYTASVKVTDLNSLELDKGNFVVDQMCEIGKPEPLNEEEARGVVE
NYNDEEVSIRVGGNTQPSKVLNKKNVEAIGLLGGQKSKADYELYNKASN
PDKVASTAFAENRNSETSDTTGTHESDRNKESSDQTGINISGFENKISY
VVQSLKEYEGKWLLFDDSEVKVTEEKDFLNSLSPSTSPTSTPYLLFYKK
L.

USP1 is a deubiquitinating enzyme that acts as part of a complex with UAF1. USP1's “deubiquitinase activity” includes its ability to deubiquitinate as part of the USP1-UAF1 complex.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some aspects, by “reduce” or “inhibit” is meant the ability to cause an overall decrease of 20% or greater. In some aspects, by “reduce” or “inhibit” is meant the ability to cause an overall decrease of 50% or greater. In some aspects, by “reduce” or “inhibit” is meant the ability to cause an overall decrease of 75%, 85%, 90%, 95%, or greater. In some aspects, the amount noted above is inhibited or decreased over a period of time, relative to a control over the same period of time.

In some aspects, inhibiting USP1 proteins is the inhibition of one or more activities or functions of USP1 proteins. It should be appreciated that the activity or function of the one or more USP1 proteins may be inhibited in vitro or in vivo. Non-limiting examples of activities and functions of USP1 include deubiquitinase activity and formation of a complex with UAF1 and are described herein. Exemplary levels of inhibition of the activity of one or more USP1 proteins include at least 10% inhibition, at least 20% inhibition, at least 30% inhibition, at least 40% inhibition, at least 50% inhibition, at least 60% inhibition, at least 70% inhibition, at least 80% inhibition, at least 90% inhibition, and up to 100% inhibition.

The terms “individual” or “subject” are used interchangeably herein to refer to an animal, for example, a mammal, such as a human. In some instances, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some instances, the subject to receive the treatment can be a patient, designating the fact that the subject has been identified as having a disorder of relevance to the treatment, or being at particular risk of contracting the disorder.

As used herein, the terms “cancer” and “tumor” refer to or describe the physiological condition in mammals in which a population of cells are characterized by unregulated cell growth. The terms encompass solid and hematological/lymphatic cancers. Examples of cancer include but are not limited to, DNA damage repair pathway deficient cancers. Additional examples of cancer include, but are not limited to, ovarian cancer, breast cancer (including triple negative breast cancer), non-small cell lung cancer (NSCLC), and osteosarcoma. The cancer can be BRCA1 or BRCA2 wild type. The cancer can also be BRCA1 or BRCA2 mutant. The cancer can further be a chemotherapy agent resistant or refractory cancer, or a chemotherapy agent resistant or refractory BRCA1 or BRCA2-mutant cancer. The cancer can be BARD1 wild type. The cancer can also be BARD1 mutant. The cancer can be BRIP1 wild type. The cancer can also be BRIP1 mutant. The cancer can be FANCA wild type. The cancer can also be FANCA mutant. The cancer can be NBN wild type. The cancer can also be NBN mutant. The cancer can be PALB2 wild type. The cancer can also be PALB2 mutant. The cancer can be RAD51 wild type. The cancer can also be RAD51 mutant. The cancer can be RAD51B wild type. The cancer can also be RAD51B mutant. The cancer can be RAD51C wild type. The cancer can also be RAD51C mutant. The cancer can be RAD51D wild type. The cancer can also be RAD51D mutant.

In one aspect, the cancer is a cancer that is homologous recombination DNA repair positive (HRR+ or HRP). A combination of a chemotherapeutic agent and USP1 inhibitor of the present disclosure provide a better effect/outcome in cancer cells that are HRR+. Homologous recombination DNA repair (HRR) is an important mechanism by which DNA damage is repaired. Homologous recombination DNA repair is a process by which double-stranded DNA breaks and interstrand crosslinks use sister chromatid as a template for repair. DNA damage can be removed in an error-free fashion via this mechanism. Additionally, during DNA replication, HRR pathways support the recovery of stalled replication forks. Curtin, N, Nature Reviews Cancer 12: 801-817 (2012); Creeden et al., BMC Cancer 21: 1154 (2021) and Scott et al., Oncotarget 12(16):1600-1614 (2021).

As used herein, the term “loss of function” mutation refers to a mutation that results in the absence of a gene, decreased expression of a gene, or the production of a gene product (e.g. protein) having decreased activity or no activity. Loss of function mutations include for example, missense mutations, nucleotide insertions, nucleotide deletions, and gene deletions. Loss of function mutations also include dominant negative mutations. Thus, cancer cells with a loss of function mutation in a gene encoding p53 include cancer cells that contain missense mutations in a gene encoding p53 as well as cancer cells that lack a gene encoding p53.

### I. USP1 Inhibitors

USP1 inhibitors have been disclosed, for example, in WO2020/132269 and WO2022/094096, each of which is herein incorporated by reference in its entirety.

In some aspects, the ubiquitin-specific-processing protease 1 (USP1) inhibitor of the present disclosure has Formula I:

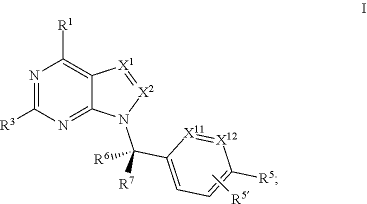

or a pharmaceutically acceptable salt or solvate thereof, wherein:
each of X¹ and X² is independently selected from N and CR²;
each of R¹ and R² is independently selected from hydrogen, halo, cyano, optionally substituted alkyl, optionally substituted alkenyl, and optionally substituted alkynyl;
R³ is an optionally substituted phenyl, optionally substituted pyridyl, optionally substituted pyrimidinyl, optionally substituted pyrazinyl, optionally substituted pyridazinyl, or optionally substituted pyrazolyl;
each of X¹¹ and X¹² is independently selected from N and CH;
R^{5′} is selected from hydrogen, optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷ optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted O-heterocyclo, optionally substituted —O-heterocyclo-(C₁-C₂) alkyl;
R⁵ is selected from optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted —O-heterocyclo, optionally substituted O-heterocyclo-(C₁-C₂) alkyl; or
one of R⁵ and one of R^{5′} on adjacent atoms are taken together with the atoms to which they are attached to form an optionally substituted (C₆-C₁₄) aryl ring; or one of R⁵ and one of R^{5′} on adjacent atoms are taken together with the atoms to which they are attached to form an optionally substituted heteroaryl ring; or one of R⁵ and one of R^{5′} on adjacent atoms are taken together with the atoms to which they are attached to form an optionally substituted (C₃-C₈) cycloalkyl ring; or one of R⁵ and one of R^{5′} on adjacent atoms are taken together with the atoms to which they are attached to form an optionally substituted heterocycloalkyl ring; or one of R⁵ and one of R^{5′} on adjacent atoms on the same atom to which they are attached are taken together to form an optionally substituted spirocycloalkyl ring; or one of R⁵ and one of R^{5′} on adjacent atoms on the same atom to which they are attached are taken together to form an optionally substituted spiroheterocycloalkyl ring;
each of R⁶ and R⁷ is independently selected from hydrogen, halo, cyano, optionally substituted alkyl, optionally substituted alkenyl, and optionally substituted alkynyl;
R²³ is selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, optionally substituted heterocyclo, optionally substituted heteroaryl, optionally substituted aryl, and optionally substituted cycloalkyl;
R^{31a} and R^{31b} are each independently selected from the group consisting of hydrogen, optionally substituted alkyl, optionally substituted alkenyl, optionally substituted alkynyl, hydroxyalkyl, (amino)alkyl, (alkylamino)alkyl, (dialkylamino)alkyl, alkoxyalkyl, cycloalkyl, (cycloalkyl)alkyl, (heterocyclo)alkyl, aralkyl, and (heteroaryl)alkyl; and
each of R²⁴, R²⁵, R²⁷, R^{32a}, and R^{32b} is independently selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, alkoxyalkyl, optionally substituted heterocyclo, optionally substituted heteroaryl, optionally substituted aryl, and optionally substituted cycloalkyl.

In some aspects, the USP1 inhibitor has one of the following structures:

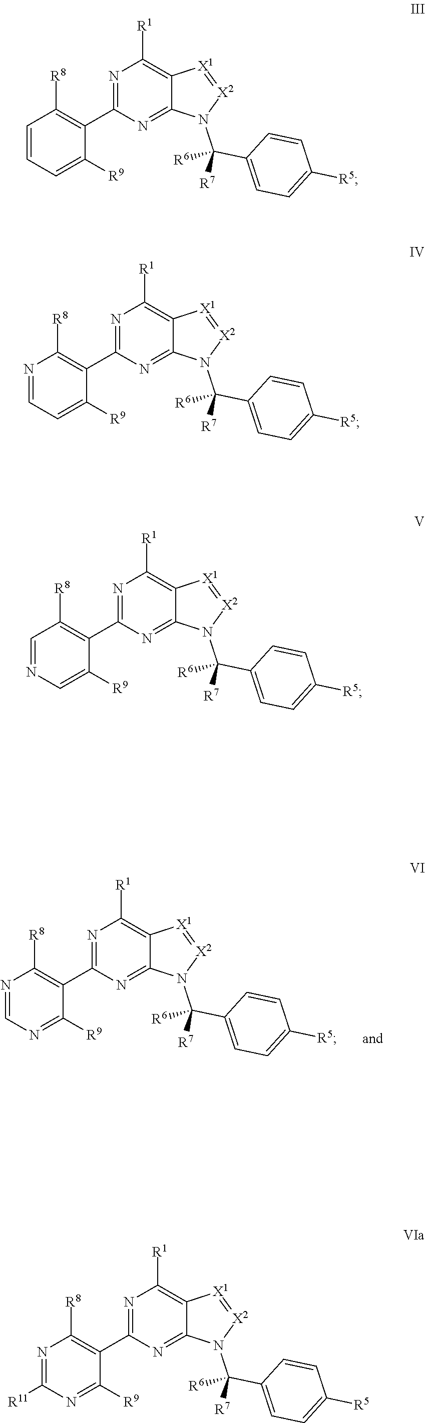

or a pharmaceutically acceptable salt or solvate thereof, wherein
each of X¹ and X² is independently selected from N and CR²;
R¹ is selected from hydrogen, halo, cyano, optionally substituted alkyl, optionally substituted alkenyl, and optionally substituted alkynyl;
R⁵ is:

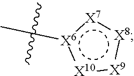

X⁶ is selected from NR¹³ and CR⁸;
X⁷ is selected from NR¹⁴ and CR⁹;
X⁸ is selected from NR¹⁵ and CR²⁰;
X⁹ is selected from NR¹⁶ and CR²¹;
X¹⁰ is selected from NR¹⁷ and CR²²;
each of R¹³, R¹⁴, R⁵, R¹⁶, and R¹⁷ is absent, or independently selected from hydrogen, halo, methyl, ethyl, isopropyl, cyclopropyl, methoxy, triazolyl, cyano, optionally substituted alkyl, amino, alkylamino, dialkylamino, difluoromethyl, trifluoromethyl, methylsulfonyl, and methylazetidinyl; and
each of R¹⁸, R¹⁹, R²⁰, R²¹, and R²² is independently selected from hydrogen, halo, methyl, ethyl, isopropyl, cyclopropyl, methoxy, triazolyl, cyano, optionally substituted alkyl, amino, alkylamino, dialkylamino, difluoromethyl, trifluoromethyl, methylsulfonyl, and methylazetidinyl;
each of R⁶ and R⁷ is independently selected from hydrogen, halo, cyano, optionally substituted alkyl, optionally substituted alkenyl, and optionally substituted alkynyl; and
each of R⁸, R⁹ and R¹¹ is independently selected from the group consisting of hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, or (heteroaryl)alkyl.

In some aspects, the USP1 inhibitor is a compound selected from the group consisting of:
(a) Compound 1:

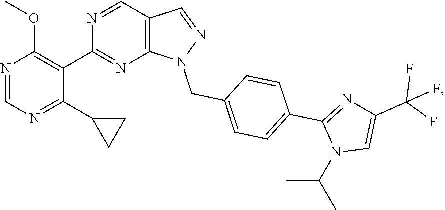 [SMILES: COc1ncnc(C2CC2)c1-c1ncc2cnn(Cc3ccc(-c4nc(C(F)(F)F)cn4C(C)C)cc3)c2n1]

(b) Compound 2:

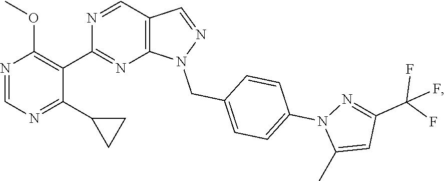 [SMILES: COc1ncnc(C2CC2)c1-c1ncc2cnn(Cc3ccc(-n4nc(C(F)(F)F)cc4C)cc3)c2n1]

(c) Compound 3:

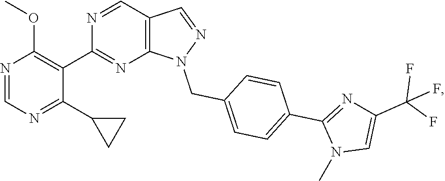 [SMILES: COc1ncnc(C2CC2)c1-c1ncc2cnn(Cc3ccc(-c4nc(C(F)(F)F)cn4C)cc3)c2n1]

and a pharmaceutically acceptable salt, hydrate, solvate, amorphous solid, or polymorph thereof.

In some aspects, the USP1 inhibitor is a compound selected from the group consisting of:
(a) Compound 1:

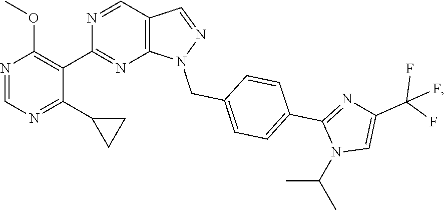 [SMILES: COc1ncnc(C2CC2)c1-c1ncc2cnn(Cc3ccc(-c4nc(C(F)(F)F)cn4C(C)C)cc3)c2n1]

(b) Compound 2:

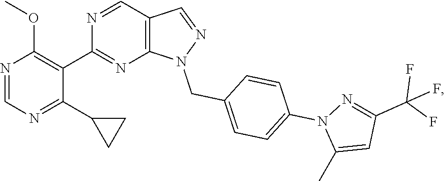 [SMILES: COc1ncnc(C2CC2)c1-c1ncc2cnn(Cc3ccc(-n4nc(C(F)(F)F)cc4C)cc3)c2n1]

(c) Compound 3:

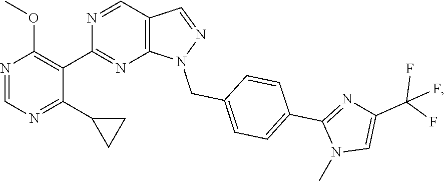 [SMILES: COc1ncnc(C2CC2)c1-c1ncc2cnn(Cc3ccc(-c4nc(C(F)(F)F)cn4C)cc3)c2n1]

and a pharmaceutically acceptable salt, hydrate, solvate, amorphous solid, polymorph, or cocrystal thereof.

The chemical name for Compound 1 is 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, as described in U.S. Publication No. US20210115049A1.

The chemical name for Compound 2 is 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine, as described in U.S. Publication No. US20210115049A1.

The chemical name for Compound 3 is 6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine, as described in U.S. Publication No. US20210115049A1.

In one aspect, the USP1 inhibitor is Compound 1, or a pharmaceutically acceptable salt, hydrate, solvate, amorphous solid, or polymorph thereof. In one aspect, the USP1 inhibitor is Compound 1, or a pharmaceutically acceptable salt, hydrate, solvate, amorphous solid, polymorph, or cocrystal thereof.

In some aspects, a USP1 inhibitor for the uses and methods provided herein is a solid state form that is a pharmaceutically acceptable salt of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound 1).

In some aspects, a USP1 inhibitor for the uses and methods provided herein is a solid state form that is a cocrystal of a pharmaceutically acceptable acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound 1). In some aspects, the cocrystal is formed between 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound 1) and a pharmaceutically acceptable acid selected from the group consisting of 1-hydroxy-2-naphthoic acid, 4-aminosalicylic acid, ascorbic acid, adipic acid, L-aspartic acid, benzene sulfonic acid, benzoic acid, trans-cinnamic acid, citric acid, ethanedisulfonic acid, fumaric acid, galactaric acid, gentisic acid, gluconic acid, D-glucuronic acid, glutamic acid, glutaric acid, glycolic acid, hexanoic acid, hippuric acid, hydrobromic acid, hydrochloric acid, lactic acid, maleic acid, L-malic acid, malonic acid, R-mandelic acid, methanesulfonic acid, mucic acid, naphthalene sulfonic acid, nicotinic acid, oxalic acid, palmitic acid, p-toluene sulfonic acid, phosphoric acid, propionic acid, saccharin, salicylic acid, stearic acid, succinic acid, sulfuric acid, L-tartaric acid, vanillic acid, vanillin, ethyl maltol, gallic acid, gallic acid ethyl ester, 4-hydroxybenzoic acid, 4-hydroxybenzoic acid methyl ester, 3,4,5-trihydroxybenzoic acid, nicotinamide, L-proline, and D-sorbitol. In some embodiments, the pharmaceutically acceptable acid is selected from the group consisting of gentisic acid, benzoic acid, salicylic acid, and gallic acid. In some aspects, the pharmaceutically acceptable acid is gentisic acid.

In some aspects, the cocrystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound 1 gentisic acid cocrystal) is an amorphous form. In some aspects, the amorphous form is substantially free of other polymorphic forms. In some aspects, the amorphous form has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In some aspects, the cocrystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound 1 gentisate) is a solid state crystalline form.

In some aspects, the cocrystal of gentisic acid and 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (Compound 1 gentisic acid cocrystal) is crystalline Form 2 (Compound 1 gentisic acid cocrystal Form 2 or “Form 2”). Compound 1 gentisic acid cocrystal, Form 2 is characterized by an XRPD pattern having peaks at 16.6±0.2, 18.7 0.2, and 22.5±0.2 degrees two theta.

In some aspects, crystalline Form 2 is an anhydrate.

In some aspects, the melting point of crystalline Form 2 is from about 184° C. to about 190° C. In some aspects, the melting point of crystalline Form 2 is from about 186° C. to about 188° C. In some aspects, the melting point of crystalline Form 2 is about 187° C.

In some aspects, crystalline Form 2 is characterized by an XRPD pattern having peaks at 16.6±0.2, 18.7±0.2, and 22.5±0.2 degrees two theta when measured by Cu Kα radiation. In another aspect, crystalline Form 2 is characterized by an XRPD pattern having peaks at 16.6±0.2, 18.7±0.2, 22.3±0.2, and 22.5±0.2 degrees two theta when measured by Cu Kα radiation. In some aspects, crystalline Form 2 is characterized by an XRPD pattern having peaks at 16.6±0.2, 18.7±0.2, 22.3±0.2, 22.5±0.2, and 26.0±0.2 degrees two theta when measured by Cu Kα radiation. In some aspects, crystalline Form 2 is characterized by an XRPD pattern having peaks at 16.6±0.2, 18.7±0.2, 20.8±0.2, 22.3±0.2, 22.5±0.2, and 26.0±0.2 degrees two theta when measured by Cu Kα radiation.

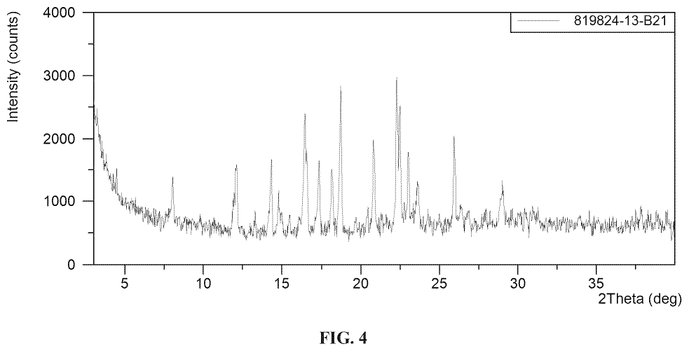

In some aspects, crystalline Form 2 is characterized by an XRPD pattern substantially as shown in FIG. 4. In some aspects, crystalline Form 2 is characterized by three or more, four or more, five or more, or six or more XRPD peaks listed in Table 2 (a and b) below:

TABLE 2a
Select XRPD Peaks for Crystalline Form 2.

| Pos. [°2Th.] | d-spacing [Å] | Rel. Int. [%] |
|---|---|---|
| 8.06 | 10.97 | 21.93 |
| 12.12 | 7.30 | 23.48 |
| 13.30 | 6.66 | 9.94 |
| 14.31 | 6.19 | 37.35 |
| 14.95 | 5.93 | 21.37 |
| 15.47 | 5.73 | 12.00 |
| 16.42 | 5.40 | 38.25 |
| 16.58 | 5.35 | 59.48 |
| 17.35 | 5.11 | 18.11 |
| 18.18 | 4.88 | 30.28 |
| 18.72 | 4.74 | 61.10 |
| 19.68 | 4.51 | 5.01 |
| 20.40 | 4.35 | 6.62 |
| 20.80 | 4.27 | 37.19 |
| 22.29 | 3.99 | 95.77 |
| 22.49 | 3.95 | 100.00 |
| 23.00 | 3.87 | 21.16 |
| 23.65 | 3.76 | 19.85 |
| 25.96 | 3.43 | 38.67 |
| 26.40 | 3.38 | 7.96 |
| 26.88 | 3.32 | 8.15 |
| 28.95 | 3.08 | 8.97 |
| 30.06 | 2.97 | 6.51 |

TABLE 2b
XRPD Peaks for Crystalline Form 2

| Pos. | d-spacing | Rel. Int. |
|---|---|---|
| [°2 Th.] | [Å] | [%] |
| 8.06 | 10.97 | 21.93 |
| 12.12 | 7.30 | 23.48 |
| 13.30 | 6.66 | 9.94 |
| 14.31 | 6.19 | 37.35 |
| 14.95 | 5.93 | 21.37 |
| 15.47 | 5.73 | 12.00 |
| 16.42 | 5.40 | 38.25 |
| 16.58 | 5.35 | 59.48 |
| 17.35 | 5.11 | 18.11 |
| 18.18 | 4.88 | 30.28 |
| 18.72 | 4.74 | 61.10 |
| 19.68 | 4.51 | 5.01 |
| 20.40 | 4.35 | 6.62 |
| 20.80 | 4.27 | 37.19 |
| 22.29 | 3.99 | 95.77 |
| 22.49 | 3.95 | 100.00 |
| 23.00 | 3.87 | 21.16 |
| 23.65 | 3.76 | 19.85 |
| 25.96 | 3.43 | 38.67 |
| 26.40 | 3.38 | 7.96 |
| 26.88 | 3.32 | 8.15 |
| 28.95 | 3.08 | 8.97 |
| 30.06 | 2.97 | 6.51 |
| 31.12 | 2.87 | 2.70 |
| 34.18 | 2.62 | 1.65 |

In some aspects, crystalline Form 2 is characterized by an endothermic peak at from about 181° C. to about 191° C., or from about 183° C. to about 189° C., or from about 185° C. to about 187° C., as determined by DSC. In some aspects, crystalline Form 2 is characterized by an endothermic peak at about 186.0° C., as determined by DSC.

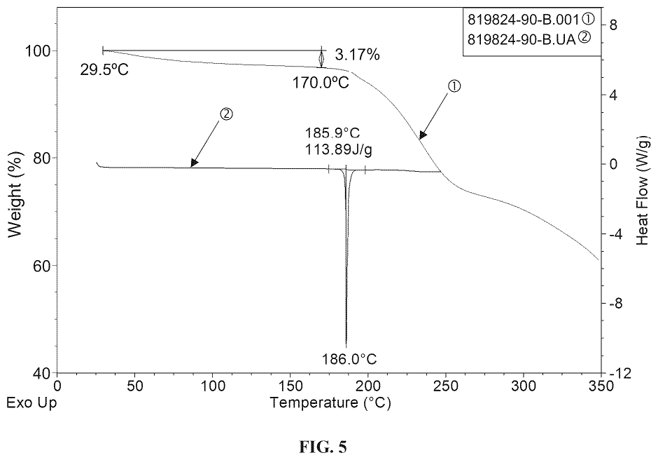

In some aspects, crystalline Form 2 is characterized by a DSC profile substantially as shown in FIG. 5.

In some aspects, crystalline Form 2 is characterized by from an about 2.5 wt % to an about 3.5 wt % loss between room temperature and about 170° C. In some aspects, crystalline Form 2 is characterized by from an about 3.0 wt % to an about 3.4 wt % loss between room temperature and about 170° C. In some aspects, crystalline Form 2 is characterized by an about 3.17 wt % loss between room temperature and about 170° C.

In some aspects, crystalline Form 2 is characterized by a TGA profile substantially as shown in FIG. 5.

In some aspects, crystalline Form 2 is characterized by at least two of the following: a) an XRPD pattern as shown in FIG. 4; b) a DSC profile as shown in FIG. 5; or c) a TGA profile as shown in FIG. 5.

In some aspects, crystalline Form 2 has a unit cell that indexes as monoclinic.

In some aspects, crystalline Form 2 has a unit cell with an a value of about 11.113 Å, a b value of about 12.356 Å, and a c value of about 24.048 Å. In some aspects, Form 2 has a unit cell with a volume of about 3223.93 Å³.

The unit cell parameters for crystalline Form 2 shown in Table 3.

TABLE 3
Unit Cell Parameters for Crystalline Form 2.

| Crystal System | Monoclinic |
|---|---|
| a [Å] | 11.113 |
| b [Å] | 12.356 |
| c [Å] | 24.048 |
| α [deg] | 90 |
| β [deg] | 102.48 |
| γ [deg] | 90 |
| Volume [Å³] | 3223.93 |
| Z CalMolated density [g/cm³] | 4, 1.419 |
| Crystal Size [mm³] | 0.31 × 0.15 × 0.11 |
| Space Group(s) | P2_{1/c} |

In some aspects, crystalline Form 2 is substantially free of other polymorphic forms. In some aspects, crystalline Form 2 has a polymorphic purity of at least 70%, or at least 75%, or at least 80%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In some aspects, the a USP1 inhibitor comprises a mixture comprising crystalline Form 2 and a second solid state form of Compound 1. In some aspects, the second solid state form of Compound 1 is crystalline Form A. In some aspects, a USP1 inhibitor comprises a mixture comprising a majority of crystalline Form 2 as compared to other solid state forms of Compound 1. Suitable solid state forms of Compound 1 are described in WO2022/094096, which is incorporated by reference herein.

In another aspect, the USP1 inhibitor is a solid state form of Compound 2, or a pharmaceutically acceptable salt, hydrate, solvate, amorphous solid, or polymorph thereof. In another aspect, the USP1 inhibitor is a solid state form of Compound 2, or a pharmaceutically acceptable salt, hydrate, solvate, amorphous solid, polymorph, or cocrystal thereof.

In some aspects, a USP1 inhibitor for the uses and methods provided herein is a solid state form that is a pharmaceutically acceptable salt of 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine (Compound 2).

In some aspects, a USP1 inhibitor for the uses and methods provided herein is a solid state form that is a cocrystal of a pharmaceutically acceptable acid and 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine (Compound 2). In some aspects, the cocrystal is formed between 6-(4-cyclopropyl-6-methoxypyrimidin-5-yl)-1-(4-(5-methyl-3-(trifluoromethyl)-1H-pyrazol-1-yl)benzyl)-1H-pyrazolo[3,4-d]pyrimidine (Compound 2) and a pharmaceutically acceptable acid selected from the group consisting of 1-hydroxy-2-naphthoic acid, 4-aminosalicylic acid, ascorbic acid, adipic acid, L-aspartic acid, benzene sulfonic acid, benzoic acid, trans-cinnamic acid, citric acid, ethanedisulfonic acid, fumaric acid, galactaric acid, gentisic acid, gluconic acid, D-glucuronic acid, glutamic acid, glutaric acid, glycolic acid, hexanoic acid, hippuric acid, lactic acid, maleic acid, L-malic acid, malonic acid, R-mandelic acid, mucic acid, naphthalene sulfonic acid, nicotinic acid, oxalic acid, palmitic acid, p-toluene sulfonic acid, phosphoric acid, propionic acid, saccharin, salicylic acid, stearic acid, succinic acid, sulfuric acid, L-tartaric acid, vanillic acid, vanillin, ethyl maltol, gallic acid, gallic acid ethyl ester, 4-hydroxybenzoic acid, 4-hydroxybenzoic acid methyl ester, 3,4,5-trihydroxybenzoic acid, nicotinamide, L-proline, and D-sorbitol. In some embodiments, the pharmaceutically acceptable acid is selected from the group consisting of gentisic acid, benzoic acid, salicylic acid, and gallic acid.

Suitable solid state forms of Compound 2 are described in WO2022/094096, which is incorporated by reference herein.

In another aspect, the USP1 inhibitor is Compound 3, or a pharmaceutically acceptable salt, hydrate, solvate, amorphous solid, or polymorph thereof. In another aspect, the USP1 inhibitor is Compound 3, or a pharmaceutically acceptable salt, hydrate, solvate, amorphous solid, polymorph, or cocrystal thereof.

In some aspects, a USP1 inhibitor for the uses and methods provided herein is a solid state form that is a pharmaceutically acceptable salt of 6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine (Compound 3).

In some aspects, a USP1 inhibitor for the uses and methods provided herein is a solid state form that is a cocrystal of a pharmaceutically acceptable acid and 6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine (Compound 3). In some aspects, the cocrystal is formed between 6-(4-cyclopropyl-6-methoxypyrimidin-5-yl)-1-(4-(1-methyl-4-(trifluoromethyl)-1H-imidazol-2-yl)benzyl)-1H-pyrazolo[3,4-d]pyrimidine (Compound 3) and a pharmaceutically acceptable acid selected from the group consisting of 1-hydroxy-2-naphthoic acid, 4-aminosalicylic acid, ascorbic acid, adipic acid, L-aspartic acid, benzene sulfonic acid, benzoic acid, trans-cinnamic acid, citric acid, fumaric acid, galactaric acid, gentisic acid, gluconic acid, D-glucuronic acid, glutamic acid, glutaric acid, glycolic acid, hexanoic acid, hippuric acid, lactic acid, maleic acid, L-malic acid, malonic acid, R-mandelic acid, mucic acid, naphthalene sulfonic acid, nicotinic acid, oxalic acid, palmitic acid, p-toluene sulfonic acid, phosphoric acid, propionic acid, saccharin, salicylic acid, stearic acid, succinic acid, sulfuric acid, L-tartaric acid, vanillic acid, vanillin, ethyl maltol, gallic acid, gallic acid ethyl ester, 4-hydroxybenzoic acid, 4-hydroxybenzoic acid methyl ester, 3,4,5-trihydroxybenzoic acid, nicotinamide, L-proline, and D-sorbitol. In some embodiments, the pharmaceutically acceptable acid is selected from the group consisting of gentisic acid, benzoic acid, salicylic acid, and gallic acid.

Suitable solid state forms of Compound 3 are described in WO2022/094096, which is incorporated by reference herein.

The present disclosure encompasses the preparation and use of salts of the USP1 inhibitors, including non-toxic pharmaceutically acceptable salts. Examples of pharmaceutically acceptable addition salts include inorganic and organic acid addition salts and basic salts. Pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. The term “pharmaceutically acceptable salt” as used herein, refers to any salt, e.g., obtained by reaction with an acid or a base, of a USP1 inhibitor of the disclosure that is physiologically tolerated in the target patient (e.g., a mammal, e.g., a human).

Acid addition salts can be formed by mixing a solution of the particular USP1 inhibitor with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the USP1 inhibitor of the present disclosure with a solution of a pharmaceutically acceptable non-toxic base such as sodium hydroxide, potassium hydroxide, choline hydroxide, sodium carbonate and the like.

In some aspects of the present disclosure, a pharmaceutically acceptable salt is formed between a compound of Formula I, Formula III, Formula IV, Formula V, Formula VI, Formula VIa, Compound 1, Compound 2, or Compound 3 and a pharmaceutically acceptable acid. In some aspects, the pharmaceutically acceptable acid is selected from the group consisting of 1-hydroxy-2-naphthoic acid, 4-aminosalicylic acid, ascorbic acid, adipic acid, L-aspartic acid, benzene sulfonic acid, benzoic acid, trans-cinnamic acid, citric acid, ethanedisulfonic acid, fumaric acid, galactaric acid, gallic acid, gentisic acid, gluconic acid, D-glucuronic acid, glutamic acid, glutaric acid, glycolic acid, hexanoic acid, hippuric acid, hydrobromic acid, hydrochloric acid, lactic acid, maleic acid, L-malic acid, malonic acid, R-mandelic acid, methanesulfonic acid, mucic acid, naphthalene sulfonic acid, nicotinic acid, oxalic acid, palmitic acid, p-toluene sulfonic acid, phosphoric acid, propionic acid, saccharin, salicylic acid, stearic acid, succinic acid, sulfuric acid, L-tartaric acid, vanillic acid, and vanillin. In some aspects, the pharmaceutically acceptable acid is selected from the group consisting of benzoic acid, gallic acid, gentisic acid and salicylic acid.

The present disclosure encompasses the preparation and use of solvates of the USP1 inhibitor. Solvates typically do not significantly alter the physiological activity or toxicity of the compounds, and as such may function as pharmacological equivalents. The term “solvate” as used herein is a combination, physical association and/or solvation of a USP1 inhibitor of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. USP1 inhibitors of the present disclosure can be present as solvated forms with a pharmaceutically acceptable solvent, such as water, methanol, ethanol, and the like, and it is intended that the present disclosure includes both solvated and unsolvated forms of the USP1 inhibitor of the present disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a USP1 inhibitor of the present disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

In some aspects of the present disclosure, the USP1 inhibitor and/or the chemotherapy agent is deuterated. In some aspects, the USP1 inhibitor and/or the chemotherapy agent are partially or completely deuterated, i.e., one or more hydrogen atoms are replaced with deuterium atoms.

The term “amorphous” as applied to a compound refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”).

The term “polymorph” as used herein refers to a crystalline form of a compound or a salt, hydrate, or solvate thereof, in a particular crystal packing arrangement. All polymorphs have the same elemental composition. The term “crystalline,” as used herein, refers to a solid state form which consists of orderly arrangement of structural units. Different crystalline forms of the same compound, or a salt, cocrystal, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility. See, e.g., Remington's Pharmaceutical Sciences, 18^{th} ed., Mack Publishing, Easton PA, 173 (1990); The United States Pharmacopeia, 23^{rd} ed., 1843-1844 (1995) (incorporated herein by reference).

Crystalline forms are most commonly characterized by X-ray powder diffraction (XRPD). An XRPD pattern of reflections (peaks, typically expressed in degrees 2-theta) is commonly considered a fingerprint of a particular crystalline form. The relative intensities of the XRPD peaks can widely vary depending on, inter alia, the sample preparation technique, crystal size distribution, filters, the sample mounting procedure, and the particular instrument employed. In some aspects, new peaks may be observed or existing peaks may disappear, depending on the type of instrument or the settings. In some aspects, any particular peak in an XRPD pattern may appear as a singlet, doublet, triplet, quartet, or multiplet, depending on the type of instrument or the settings, the sensitivity of the instrument, measuring conditions, and/or purity of the crystalline form. In some aspects, any particular peak in an XRPD may appear in a symmetric shape or in an asymmetric shape, e.g., having a shoulder. A skilled artisan understanding these variations is capable of discriminating or ascertaining the defining features or characteristics of a particular crystal form using XRPD, as well as using other known physicochemical techniques.

In various aspects, the USP1 inhibitors reduce the level of USP1 protein and/or inhibit or reduce at least one biological activity of USP1 protein.

In some aspects, the USP1 inhibitors specifically bind to USP1 protein. In some aspects, the USP1 inhibitors specifically bind to USP1 protein in a USP1-UAF1 complex. In some aspects, the USP1 inhibitors specifically bind to USP1 mRNA. In some aspects, the USP1 inhibitors specifically bind to USP1 protein (alone or in a USP1-UAF1 complex) or USP1 mRNA. In some aspects, the USP1 inhibitors specifically bind to UAF1 (alone or in a USP1-UAF1 complex) and inhibit or reduces formation or activity of the USP1-UAF1 complex.

In some aspects, the USP1 inhibitors decrease the formation of the USP1-UAF1 complex. In some aspects, the USP1 inhibitors decrease the activity of the USP1-UAF1 complex. In some aspects, the USP1 inhibitors decrease the deubiquitinase activity of USP1. In some aspects, the USP1 inhibitors increase mono-ubiquitinated PCNA. In some aspects, the USP1 inhibitors increase mono-ubiquitinated FANCD2. In some aspects, the USP1 inhibitors increase mono-ubiquitinated FANCI.

In some aspects, the USP1 inhibitors do not bind to other deubiquitinases, other USP proteins, or other UAF1 complexes (e.g., USP46-UAF1) or bind deubiquitinases, other USP proteins, or other UAF1 complexes (e.g., USP46-UAF1) with at least 5-fold, at least 10-fold, at least 20-fold, or at least 100-fold reduced affinity compared to the affinity for USP1 (i.e., the K_{D} of the USP1 inhibitor for other deubiquitinases, other USP proteins, or other UAF1 complexes (e.g., USP46-UAF1) is at least 5-fold, at least 10-fold, at least 20-fold, or at least 100-fold higher than the K_{D} for USP1).

In some aspects, the USP1 inhibitors inhibit USP1 deubiquitinase activity with an IC50 of less than about 50 nM, between about 50 nM and about 200 nM, between about 200 nM and about 2 pM, or greater than 2 pM, e.g., as measured using the assay disclosed in U.S. Patent Application Publication No. 2017/0145012 or IC50 of 50 nM to 1000 nM, e.g., as measured using the assay disclosed in Liang et al., Nat Chem Biol 10: 289-304 (2014). In some aspects, the USP1 inhibitors inhibit USP1 deubiquitinase activity with an IC50 as measured using the assay disclosed in Chen, et al., Chem Biol., 18(11):1390-1400 (2011). In some aspects, the USP1 inhibitors do not inhibit the activity of other deubiquitinases, other USP proteins, or other UAF1 complexes (e.g., USP46-UAF1) or inhibit the activity of other deubiquitinases, other USP proteins, or other UAF1 complexes (e.g., USP46-UAF1) with at least 5-fold, at least 10-fold, at least 20-fold, or at least 100-fold higher IC50 compared to the IC50 for inhibition of USP1 deubiquitinase activity.

In some aspects, the USP1 inhibitors of the present disclosure bind to a USP1 protein with an affinity in the range of 1 pM to 100 μM, or 1 pM to 1 μM, or 1 pM to 500 nM, or 1 pM to 100 nM. In some aspects, the USP1 inhibitors of the present disclosure bind to a USP1 protein with an affinity of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some aspects, the USP1 inhibitors of the present disclosure bind to a USP1 protein with an affinity of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some aspects, the USP1 inhibitors of the present disclosure bind to a USP1 protein with an affinity of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM.

In some aspects, the USP1 inhibitors of the present disclosure bind to a USP1 protein with an affinity of less than 1 pM, less than 500 nM, less than 100 nM, less than 10 nM, or less than 1 nM. In some aspects, the USP1 inhibitors bind to a USP1 protein with an affinity of less than 1 nM.

In some aspects, the USP1 inhibitors of the present disclosure inhibit USP1 activity with an IC₅₀ of 1 pM to 100 μM, or 1 pM to 1 μM, or 1 pM to 500 nM, or 1 pM to 100 nM. In some aspects, the USP1 inhibitors inhibit USP1 activity with an IC₅₀ of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some aspects, the USP1 inhibitors inhibit USP1 activity with an IC₅₀ of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM.

In some aspects, the USP1 inhibitors of the present disclosure inhibit USP1 activity with an IC₅₀ of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some aspects, the USP1 inhibitors inhibit USP1 activity with an IC₅₀ of less than 1 μM, less than 500 nM, less than 100 nM, less than 10 nM, or less than 1 nM. In some aspects, the USP1 inhibitors inhibit USP1 activity with an IC₅₀ of less than 1 nM.

### Exemplary Assays for Inhibition of USP1

Any suitable assay in the art can be used to determine an activity, detect an outcome or effect, or determine efficacy. Suitable assays are provided, for example, in U.S. Publication No. US20210115049A1.

In some instances, a method of determining whether a USP1 inhibitor compound inhibits USP1 deubiquitinase activity measures a change in mass upon di-ubiquitin cleavage of deubiquitinase binding. For example, ubiquitin aldehyde and ubiquitin vinyl sulfone form covalent irreversible linkages to deubiquitinases that result in observable mass changes to the deubiquitinases. Similarly, cleavage of di-ubiquitins results in an observable mass change.

In some instances, a method of determining whether a USP1 inhibitor compound inhibits USP1 deubiquitinase activity involves an increase in luminescence or fluorescence upon cleavage, e.g., that can be monitored on a plate reader. Such assays can use ubiquitin linked to a flurophore through a linker linkage, such as ubiquitin-7-amino-4-methylcoumarin (Ub-AMC) or ubiquitin-Rhodamine110. Such assays can also use a di-ubiquitin containing an isopeptide linkage. Exemplary di-ubiquitins can comprise a flurophore on one ubiquitin and a quencher on the other ubiquitin such that fluorescence increases with then di-ubiquitin is cleaved. Such assays can also use enzyme-coupled systems wherein ubiquitin is coupled to an enzyme that is only active in producing a fluorescence enzyme product when released from the ubiquitin.

### II. Chemotherapy Agents

In some aspects, the chemotherapy agent of the present disclosure is a platin, a monofunctional alkylator, a bifunctional alkylator, an antimetabolite, a topoisomerase inhibitor, or a combination thereof.

In some aspects, the chemotherapy agent is a platin.

In some aspects, the platin is selected from the group consisting of cisplatin, carboplatin, oxaliplatin, satraplatin, picoplatin, nedaplatin, and triplatin.

In some aspects, the chemotherapy agent is cisplatin or carboplatin.

In some aspects, the chemotherapy agent is a monofunctional alkylator. In some aspects, the monofunctional alkylator is selected from the group consisting of an alkylsulfonate, a nitrosourea, and temozolomide.

In some aspects, the chemotherapy agent is a bifunctional alkylator. In some aspects, the bifunctional alkylator is selected from the group consisting of nitrogen mustard and mitomycin C.

In some aspects, the chemotherapy agent is an antimetabolite. In some aspects, the antimetabolite is selected from the group consisting of gemcitabine, 5-fluorouracil, a thiopurine, and a folate analog.

In some aspects, the chemotherapy agent is a topoisomerase inhibitor. In some aspects, the topoisomerase inhibitor is selected from the group consisting of a campothecin, an anthracycline, and an etoposide.

In various aspects, the platins of the present disclosure are platinum containing compounds. Platinum containing compounds include agents such as cisplatin, carboplatin, oxaliplatin, satraplatin, picoplatin, nedaplatin and triplatin. Platinum containing compounds cause crosslinking of DNA as monoadduct, interstrand crosslinks, intrastrand crosslinks or DNA protein crosslinks. The resulting crosslinking inhibits DNA repair and/or DNA synthesis in cancer cells. These compounds are sometimes described as being alkylating-like agents despite the fact that they do not have an alkyl group. Cisplatin was the first platinum containing compound to be discovered and was first approved by the U.S. Food and Drug Administration in 1978. Carboplatin was introduced in the 1980s and has been demonstrated to have lower side-effects than cisplatin in ovarian cancer and lung cancer (Hartmann and Lipp, Exper. Opin. Pharmacother. 2003, 4(6) 889-901).

In some aspects, the platin is cisplatin, carboplatin, oxaliplatin, satraplatin, picoplatin, nedaplatin or triplatin. In some aspects, the platin is nedaplatin, cisplatin, carboplatin or oxaliplatin. In some aspects, the platin is cisplatin, carboplatin or oxaliplatin. In some aspects, the platin is cisplatin or carboplatin. In some aspects, the platin is cisplatin. In some aspects, the platin is carboplatin.

In various aspects, the alkylators of the present disclosure are alkylating agents used in cancer treatment that attach alkyl groups to DNA. Because cancer cells, in general, proliferate faster and with less error-correcting than healthy cells, cancer cells are more sensitive to DNA damage, such as being alkylated. In some aspects, the alkylating agents are active under conditions present in cells. In some aspects, the alkylating agents stop tumor growth by crosslinking guanine nucleobases in DNA double-helix strands, directly attacking DNA. In some aspects, the alkylating agents can be monofunctional alkylators or bifunctional alkylators. In some aspects, the monofunctional alkylators can react only with one guanine residue of DNA. In some aspects, the bifunctional alkylators can react with two different guanine residues of DNA.

In some aspects, the monofunctional alkylator is an alkylsulfonate, a nitrosourea, or a temozolomide. In some aspects, the monofunctional alkylator is an alkylsulfonate. In some aspects, the monofunctional alkylator is a nitrosourea. In some aspects, the monofunctional alkylator is a temozolomide. In some aspects, the bifunctional alkylator is nitrogen mustard, or mitomycin C. In some aspects, the bifunctional alkylator is nitrogen mustard. In some aspects, the bifunctional alkylator is mitomycin C.

In various aspects, the antimetabolites of the present disclosure are chemicals that inhibit the use of a metabolite. In some aspects, the antimetabolites are often similar in structure to the metabolite that they interfere with. Thus, competitive inhibition can occur, and the presence of antimetabolites can have toxic effects on cells, such as halting cell growth and cell division. In some aspects, the antimetabolites are used in cancer treatment. In some aspects, the antimetabolites interfere with DNA production and therefore cell division and tumor growth. In some aspects, the antimetabolites impair DNA replication, either by incorporation of chemically altered nucleotides or by depleting the supply of deoxynucleotides needed for DNA replication and cell proliferation.

In some aspects, the antimetabolite is gemcitabine, 5-fluorouracil, a thiopurine, a folate analog, a capecitabine, a cytarabine, a floxuridine, a fludarabine, or a hydroxycarbamide. In some aspects, the antimetabolite is gemcitabine, 5-fluorouracil, a thiopurine, or a folate analog. In some aspects, the antimetabolite is gemcitabine. In some aspects, the antimetabolite is 5-fluorouracil. In some aspects, the antimetabolite is a thiopurine. In some aspects, the antimetabolite is folate analog including a methotrexate, a pemetrexed, a phototrexate, a proguanil, a pyrimethamine, a trimethoprim, and leucovorin calcium.

In various aspects, the topoisomerase inhibitors of the present disclosure are chemical compounds that block the action of topoisomerases. Topoisomerase plays important roles in cellular reproduction and DNA organization. Topoisomerase mediates the cleavage of single and double stranded DNA to relax supercoils, untangles catenanes, and condenses chromosomes in eukaryotic cells. In some aspects, the topoisomerase inhibitors prevent topoisomerases from performing DNA strand breaks. In some aspects, the topoisomerase inhibitors associate with topoisomerase-DNA complexes and prevent the re-ligation step of the topoisomerase mechanism. These topoisomerase-DNA-inhibitor complexes are cytotoxic agents, as the un-repaired single- and double stranded DNA breaks they cause can lead to apoptosis and cell death. In some aspects, the topoisomerase inhibitors are used as therapeutics against infectious and cancerous cells.

In some aspects, the topoisomerase inhibitor is a campothecin, a topotecan, an irinotecan, a belotecan, an indenoisoquinoline, a phenanthridines, an indolocarbazoles, an anthracycline, an etoposide, a teniposide, a dexrazoxane, a novobiocin, or a merbarone. In some aspects, the topoisomerase inhibitor is a campothecin, an anthracycline, or an etoposide. In some aspects, the topoisomerase inhibitor is a campothecin. In some aspects, the topoisomerase inhibitor is an anthracycline. In some aspects, the topoisomerase inhibitor is an etoposide.

In some aspects, the chemotherapy agents can be a DNA damaging agent.

### III. Methods of Treatment

Since combinations of the present disclosure are (i) chemotherapy agents and (ii) ubiquitin-specific-processing protease 1 (USP1) inhibitors having Formula I, the present disclosure provides a method of damaging DNA and/or inhibiting a USP1 protein comprising administering one or more combinations of the present disclosure.

Since combinations of the present disclosure are (i) chemotherapy agents and (ii) ubiquitin-specific-processing protease 1 (USP1) inhibitors having Formula I, the present disclosure is directed generally to a method for treating a disease, condition, or disorder responsive to DNA damage and/or USP1 protein inhibition in a subject suffering from, or at risk of suffering from, the disorder, the method comprising administering to the subject an effective amount of one or more combinations of the present disclosure.

The present disclosure is further directed to a method of damaging DNA and/or inhibiting USP1 proteins in a subject in need thereof, the method comprising administering to the subject a therapeutically effective amount of a combination of the present disclosure.

In some aspects, the combinations of the present disclosure can be used to damage DNA and/or inhibit the activity of a USP1 protein. For example, in some aspects, a method of damaging DNA and/or inhibiting a USP1 protein comprises contacting the DNA and/or USP1 protein with a combination of the present disclosure. The contacting can occur in vitro or in vivo.

In some aspects, the combinations of the present disclosure can be used to treat a DNA and/or USP1 protein mediated disorder. A DNA and/or USP1 protein mediated disorder is any pathological condition in which a DNA and/or USP1 protein is known to play a role. In some aspects, a DNA and/or USP1 protein mediated disorder is a proliferative disease such as cancer. In some aspects, the combinations of the disclosure can delay, reduce, or prevent rebounding (rapid re-growth) of a tumor. In some aspects, the combination of the disclosure is not significantly more toxic than the chemotherapy agent alone. In some aspects, the combination of the disclosure is not significantly more toxic than the USP1 inhibitor alone. In some aspects, the combination of the disclosure is not significantly more toxic than either the chemotherapy agent alone or the USP1 inhibitor alone.

In some aspects, the combination of the disclosure is less toxic than the chemotherapy agent alone. In some aspects, the combination of the disclosure is less toxic than the USP1 inhibitor alone. In some aspects, the combination of the disclosure is less toxic than either the chemotherapy agent alone or the USP1 inhibitor alone.

In some aspects, the combination of the disclosure is administered in an effective amount to reduce unacceptable toxicity and/or unacceptable adverse reactions of a chemotherapy agent administered as a monotherapy. In some aspects, the combination of the disclosure is administered in an effective amount to reduce unacceptable toxicity and/or unacceptable adverse reactions of a USP1 inhibitor administered as a monotherapy. In some aspects, the combination of the disclosure is administered in an effective amount to reduce unacceptable toxicity and/or unacceptable adverse reactions of either a chemotherapy agent administered as a monotherapy or a USP1 inhibitor administered as a monotherapy. Exemplary toxicities or adverse reactions include hematological toxicity such as thrombocytopenia, anemia, or neutropenia, pneumonitis, dyspnea, fever, cough, wheezing, a radiological abnormality, hypertension, myelodysplastic syndrome/acute myeloid leukemia (MDS/AML), nausea, and/or fatigue.

Various methods of treating diseases and disorders with the combinations of the disclosure are provided herein. Exemplary diseases and disorders that may be treated with the combinations of the disclosure include, but are not limited to, cancer.

In some aspects, methods of treating cancer with combinations of the disclosure are provided. Such methods comprise administering to a subject with cancer a therapeutically effective amount of a combination of the disclosure.

In some aspects, such methods comprise (a) identifying a cancer in a subject as a chemotherapy agent and/or USP1 inhibitor-sensitive cancer and then (b) administering a therapeutically effective amount of a combination of the disclosure to the subject.

In some aspects, the cancer cells of said subject are homologous recombination repair (HRR) positive.

In some aspects, the cancer to be treated with a combination of the disclosure is an advanced solid tumor. An advanced solid tumor is a solid tumor that is unresectable and/or metastatic. In other aspects, the cancer to be treated with a combination of the disclosure is a solid tumor that is a respectable tumor, either benign or metastatic, where the combination is administered before and/or after a resection procedure.

In some aspects, the cancer to be treated with a combination of the disclosure is selected from the group consisting of lung cancer, non-small cell lung cancer (NSCLC), colon cancer, bladder cancer, osteosarcoma, ovarian cancer, skin cancer, uterine cancer, peritoneal cancer, endometrial cancer, pancreatic cancer, gastric cancer, cholangiocarcinoma, and breast cancer. In some aspects, the cancer is lung cancer. In some aspects, the cancer is non-small cell lung cancer (NSCLC). In some aspects, the cancer is colon cancer. In some aspects, the cancer is bladder cancer. In some aspects, the cancer is osteosarcoma. In some aspects, the cancer is ovarian cancer. In some aspects, the cancer is skin cancer. In some aspects, the cancer is uterine cancer. In some aspects, the cancer is peritoneal cancer. In some aspects, the cancer is endometrial cancer. In some aspects, the cancer is breast cancer.

In some aspects, the cancer to be treated with a combination of the disclosure was previously treated with a platinum therapy. In some aspects, the cancer to be treated with a combination of the disclosure is a platinum resistant cancer. In some aspects, the cancer to be treated with a combination of the disclosure is a platinum refractory cancer. In some aspects, the platinum therapy is selected from the group consisting of cisplatin, carboplatin, oxaliplatin, and combinations thereof.

In some aspects, the chemotherapy agent and the USP1 inhibitor are administered sequentially. In some aspects, the chemotherapy agent is administered first, and the USP1 inhibitor is administered second. In some aspects, the USP1 inhibitor is administered first, and the chemotherapy agent is administered second. In some aspects, the chemotherapy agent and the USP1 inhibitor are administered simultaneously.

In some aspects, the combination of the present disclosure is administered in a therapeutically effective amount sufficient to produce one or more therapeutic effects selected from the group consisting of (i) reduction in tumor size, (ii) increase in tumor regression rate, and (iii) reduction or inhibition of tumor growth. In some aspects, the combination of the present disclosure is administered in a therapeutically effective amount sufficient to produce an effect of reduction in tumor size. In some aspects, the combination of the present disclosure is administered in a therapeutically effective amount sufficient to produce an effect of increase in tumor regression rate. In some aspects, the combination of the present disclosure is administered in a therapeutically effective amount sufficient to produce an effect of reduction or inhibition of tumor growth.

In some embodiments, the chemotherapy agent is administered at the approved dose for the particular indication. In other embodiments, the chemotherapy agent is administered at any dose disclosed herein. In some embodiments, the platinum-based chemotherapy is cisplatin, carboplatin, oxaliplatin, satraplatin, picoplatin, Nedaplatin, Triplatin, Lipoplatin, or combinations thereof. In certain embodiments, the platinum-based chemotherapy is any other platinum-based chemotherapy known in the art. In some embodiments, the chemotherapy agent is the nucleotide analog gemcitabine. In an embodiment, the chemotherapy agent is a folate antimetabolite. In an embodiment, the folate antimetabolite is pemetrexed.

In some aspects, the USP1 inhibitor, gemcitabine and carboplatin are administered to a subject. In some aspects, the subject has ovarian cancer, triple negative breast cancer, or non-small cell lung cancer. In some aspects, the subject has ovarian cancer. In some aspects, the subject has triple negative breast cancer. In some aspects, the subject has non-small cell lung cancer.

In some aspects, the USP1 inhibitor, leucovorin calcium, 5-fluorouracil, and oxaliplatin are administered are administered to a subject. In some aspects, the subject has colorectal cancer, pancreatic cancer, cholangiocarcinoma, esophageal cancer, or gastric cancer. In some aspects, the subject has colorectal cancer. In some aspects, the subject has pancreatic cancer. In some aspects, the subject has cholangiocarcinoma. In some aspects, the subject has esophageal cancer. In some aspects, the subject has gastric cancer.

In some aspects, the USP1 inhibitor and the chemotherapy agent are provided in same dosage forms. In some aspects, the USP1 inhibitor is provided in an oral dosage form. In some aspects, the chemotherapy agent is provided in an oral dosage form. In some aspects, the USP1 inhibitor and the chemotherapy agent are provided in separate dosage forms. In some aspects, the USP1 inhibitor is provided in an oral dosage form. In some aspects, the chemotherapy agent is provided in an injectable dosage form.

In some aspects, the subject is a mammal. In some aspects, the mammal is a human.

In some aspects, a combination of the disclosure is used in combination with one or more additional therapeutic agents to treat cancer.

In some aspects, provided herein are combinations of the disclosure for use as a medicament or for use in preparing a medicament, e.g., for the treatment of cancer. In some aspects, provided herein are combinations of the disclosure for use in a method for the treatment of cancer.

### IV. Pharmaceutical Compositions and Kits

Combinations of the disclosure can be administered to a mammal in the form of a raw chemicals without any other components present, or combinations of the disclosure can also be administered to a mammal as part of a pharmaceutical composition containing the compound combined with a suitable pharmaceutically acceptable carrier (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Standard pharmaceutical carriers and their formulations are described in Remington's Pharmaceutical Sciences, Mack Publishing Co., Easton, PA, 19th ed. 1995. The USP1 inhibitor and the chemotherapy agent can be present in the same pharmaceutical composition; or the USP1 inhibitor and the chemotherapy agent can be in separate pharmaceutical compositions that can be administered concurrently or consecutively.

A pharmaceutical combination composition of the present disclosure may be prepared as liquid suspensions or solutions using a liquid, such as an oil, water, an alcohol, and combinations of these.

As provided herein, a USP1 inhibitor (e.g., 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine or a pharmaceutically acceptable salt, solvate, hydrate, or cocrystal thereof) can be in any pharmaceutical composition including all compositions where such an inhibitor is combined with one or more pharmaceutically acceptable carriers. In some aspects, the USP1 inhibitor is present in a composition in an amount that is effective to achieve its intended therapeutic purpose.

In some aspects, the present disclosure provides a pharmaceutical composition comprising a USP1 inhibitor (e.g., 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine or a pharmaceutically acceptable salt, solvate, hydrate, or cocrystal thereof), and a pharmaceutically acceptable carrier. In some aspects, a pharmaceutical composition comprises crystalline Form 2 of the gentisic acid cocrystal of Compound 1.

Pharmaceutical combination compositions within the scope of the present disclosure include all compositions where a USP1 inhibitor and a chemotherapy agent of the disclosure are combined with one or more pharmaceutically acceptable carriers. In one aspect, the USP1 inhibitor and chemotherapy agent of the disclosure are present in the composition in an amount that is effective to achieve its intended therapeutic purpose.

A pharmaceutical composition of the present disclosure can be orally administered in any orally acceptable dosage form including e.g., capsules, tablets, aqueous suspensions, or solutions. In some aspects, the capsule is a gelatin capsule.

For oral administration, known carriers can be included in the pharmaceutical composition. For example, microcrystalline cellulose, sodium citrate, calcium carbonate, dicalcium phosphate and glycine may be employed along with various disintegrants such as starch (preferably corn, potato or tapioca starch), methylcellulose, alginic acid and certain complex silicates, together with granulation binders such as polyvinylpyrrolidone, sucrose, gelatin and acacia, can be included in a tablet. Additionally, lubricating agents such as magnesium stearate, sodium lauryl sulfate and talc are often useful for tableting purposes. Solid compositions of a similar type may also be employed as fillers in gelatin capsules. Preferred materials in this connection include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

The pharmaceutical compositions to be used for in vivo administration can be sterile. This is readily accomplished by filtration through, e.g., sterile filtration membranes.

Pharmaceutical compositions within the scope of the present disclosure include all compositions where a USP1 inhibitor is combined with one or more pharmaceutically acceptable carriers. In some aspects, the USP1 inhibitor is present in the composition in an amount that is effective to achieve its intended therapeutic purpose.

A pharmaceutical composition of the present disclosure can be administered to any patient that may experience the beneficial effects of a Compound of the Disclosure. Foremost among such patients are mammals, e.g., humans and companion animals, although the disclosure is not intended to be so limited. In some aspects, the patient is a human. In some aspects, a pharmaceutical compositions of the present disclosure can be administered to a patient having a chemotherapy agent resistant or refractory cancer. In some aspects, a pharmaceutical compositions of the present disclosure can be administered to a patient having a chemotherapy agent resistant or refractory BRCA1-deficient cancer. In some aspects, a pharmaceutical composition of the present disclosure can be administered to a patient having a platinum resistant or platinum refractory cancer.

In some aspects, the present disclosure provides kits which comprise a USP1 inhibitor (e.g., 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine or a pharmaceutically acceptable salt, solvate, hydrate, or cocrystal thereof) or a pharmaceutical composition comprising the same packaged in a manner that facilitates its use to practice methods of the present disclosure. In some aspects, the kit includes a USP1 inhibitor packaged in a container, such as a sealed bottle or vessel, with a label affixed to the container or included in the kit that describes use of the USP1 inhibitor or pharmaceutical composition thereof to practice the method of the disclosure. In some aspects, the USP1 inhibitor is packaged in a unit dosage form. The kit further can include a device suitable for administering the USP1 inhibitor or pharmaceutical composition thereof according to the intended route of administration. In some aspects, the present disclosure provides a kit which comprises a USP1 inhibitor or pharmaceutical composition thereof, and instructions for administering the inhibitor or composition to a patient having cancer.

In some aspects, the present disclosure provides a pharmaceutical composition comprising a USP1 inhibitor (e.g., 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine or a pharmaceutically acceptable salt, solvate, hydrate, or cocrystal thereof), a chemotherapy agent, and a pharmaceutically acceptable carrier, wherein the pharmaceutical composition is for use in treating cancer (e.g., a solid tumor). In some aspects, the cancer is an advanced solid tumor.

In some aspects, the present disclosure provides a pharmaceutical composition comprising a USP1 inhibitor (e.g., 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine or a pharmaceutically acceptable salt, solvate, hydrate, or cocrystal thereof), and a chemotherapy agent and a pharmaceutically acceptable carrier, wherein the pharmaceutical composition is for the manufacture of a medicament for treatment of cancer (e.g., a solid tumor).

### V. Methods of Preparation of USP1 Inhibitors

USP1 inhibitors for uses and methods provided herein can be prepared in any way. Methods of synthesizing USP1 inhibitors are provided, for example, in WO2020/132269 and WO2022/094096, each of which is herein incorporated by reference in its entirety.

In some aspects, a USP1 inhibitor is prepared by a method comprising a) adding a suitable amount of Compound 1 to a suitable amount of a suitable solvent system to obtain a suspension; b) stirring the suspension; and c) collecting the solid product from step b). In some aspects, a suitable pharmaceutically acceptable acid is added during step a). In some aspects, the suitable solvent system is selected from the group consisting of acetonitrile, acetone, cyclohexane, dichloromethane, dimethylacetamide, dimethyl sulfoxide, ethanol, ethyl acetate, isopropyl alcohol, isopropyl acetate, methanol, methyl ethyl ketone, 4-methyl-2-pentanone, methyl tert-butyl ether, 2-methyl tetrahydrofuran, n-heptane, n-methyl pyrrolidone, tetrahydrofuran, toluene, water, and mixtures thereof. In some aspects, the suitable solvent system is selected from the group consisting of ethyl acetate, n-heptane, and mixtures thereof.

In some aspects, a USP1 inhibitor is prepared by a method comprising a) dissolving a suitable amount of Compound 1 in a suitable amount of a suitable solvent to make a solution; b) adding a suitable amount of a suitable anti-solvent; c) adding seed crystals of a solid state form of Compound 1; d) stirring the resulting suspension; and e) collecting the solid product produced from step d). In some aspects, the method further comprises adding a suitable pharmaceutically acceptable acid during step a). In some aspects, the method further comprises adding a suitable anti-solvent after step c) and before step d). In some aspects, the suitable solvent and anti-solvent are selected from the group consisting of acetonitrile, acetone, cyclohexane, dichloromethane, dimethylacetamide, dimethyl sulfoxide, ethanol, ethyl acetate, isopropyl alcohol, isopropyl acetate, methanol, methyl ethyl ketone, 4-methyl-2-pentanone, methyl tert-butyl ether, 2-methyl tetrahydrofuran, n-heptane, n-methyl pyrrolidone, tetrahydrofuran, toluene, water, and mixtures thereof. In some aspects, the suitable solvent and anti-solvent are selected from the group consisting of ethyl acetate, n-heptane, and mixtures thereof. In some aspects, the suitable solvent is ethyl acetate. In some aspects, the suitable anti-solvent is n-heptane.

In some aspects, Compound 1 is added to the suitable solvent system at a temperature of from about room temperature to about 100° C., or from about room temperature to about 75° C., or from about room temperature to about 50° C., or from about room temperature to about 40° C. In some aspects, Compound 1 is added to the suitable solvent system at about room temperature.

In some aspects, a USP1 inhibitor that is a crystalline Form 2 of a gentisic acid cocrystal of Compound 1 is prepared by a method comprising: a) adding a suitable amount of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine and gentisic acid to a suitable amount of a suitable solvent system at room temperature to obtain a suspension; b) stirring the suspension from step a); and c) collecting the solid product from step b). In some aspects, the suitable solvent system is selected from the group consisting of ethyl acetate, n-heptane, and mixtures thereof.

In some aspects, a USP1 inhibitor that is a crystalline Form 2 of a gentisic acid cocrystal of Compound 1 is prepared by a method comprising a) dissolving a suitable amount of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine and gentisic acid in a suitable amount of a suitable solvent at room temperature to make a solution; b) adding a suitable amount of a suitable anti-solvent; c) adding seed crystals of crystalline Form 2 of a gentisic acid cocrystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Compound 1; d) stirring the resulting suspension; and e) collecting the solid product produced from step d). In some aspects, the method further comprises adding a suitable anti-solvent after step c) and before step d). In some aspects, the suitable solvent is ethyl acetate. In some aspects, the suitable anti-solvent is n-heptane.

In some aspects, a USP1 inhibitor is a Crystalline Form 2 of a gentisic acid cocrystal of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine of Compound 1 prepared by any of the methods disclosed herein.

### EXAMPLES

### Example 1: Maximum Tolerated Dose of Compound 1 Cocrystal in Combination with Cisplatin in Non-Tumor Bearing Balb/c Nude Mice

The maximum tolerated dose (MTD) of the USP1 inhibitor Compound 1 cocrystal in combination with cisplatin was evaluated in non-tumor bearing Balb/c nude mice. 6-8 week old female Balb/c nude mice from Beijing Anikeeper Biotech Co. Ltd were randomized into groups of 5 for control, cisplatin (4 mg/kg) alone, cisplatin (5 mg/kg) alone, cisplatin (6 mg/kg) alone, Compound 1 (50 mg/kg) and cisplatin (4 mg/kg) combination group, Compound 1 (100 mg/kg) and cisplatin (4 mg/kg) combination group, Compound 1 (300 mg/kg) and cisplatin (4 mg/kg) combination group, Compound 1 (50 mg/kg) and cisplatin (5 mg/kg) combination group, Compound 1 (100 mg/kg) and cisplatin (5 mg/kg) combination group, Compound 1 (300 mg/kg) and cisplatin (5 mg/kg) combination group, Compound 1 (50 mg/kg) and cisplatin (6 mg/kg) combination group, Compound 1 (100 mg/kg) and cisplatin (6 mg/kg) combination group, and Compound 1 (300 mg/kg) and cisplatin (6 mg/kg) combination group. Mice were treated with the relevant dose of Compound 1 via oral gavage once daily for 28 days or relevant dose of cisplatin via intravenous injection once a week on days 0, 7, 14, and 21. Cisplatin is commonly dosed at 4 to 6 mg/kg once a week and has a reported MTD of 6 mg/kg for a single dose in Balb/c nude mice (Aston W., et al., BMC Cancer, 17:684 (2017)).

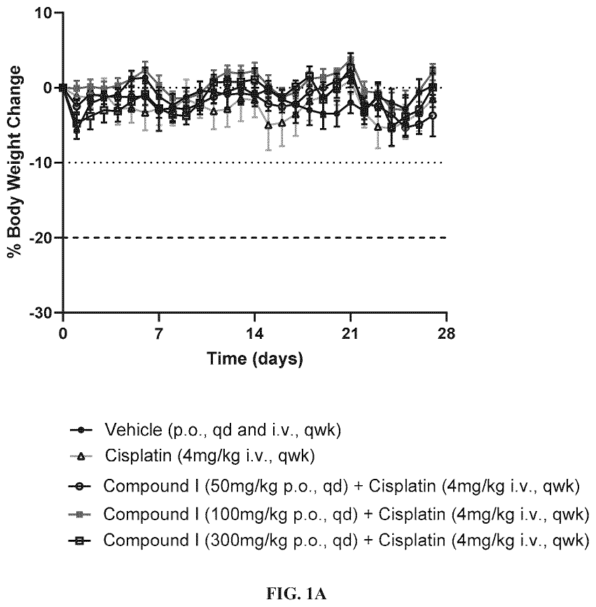

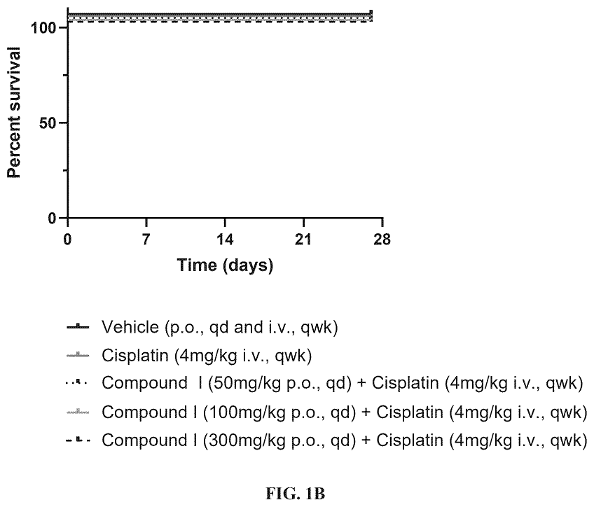

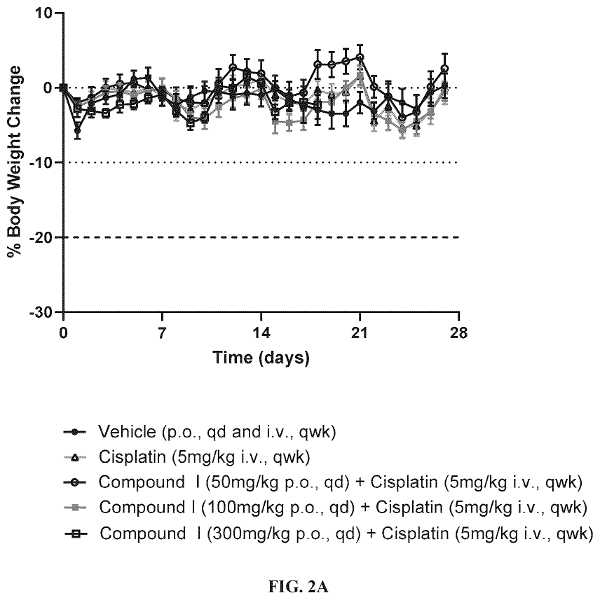

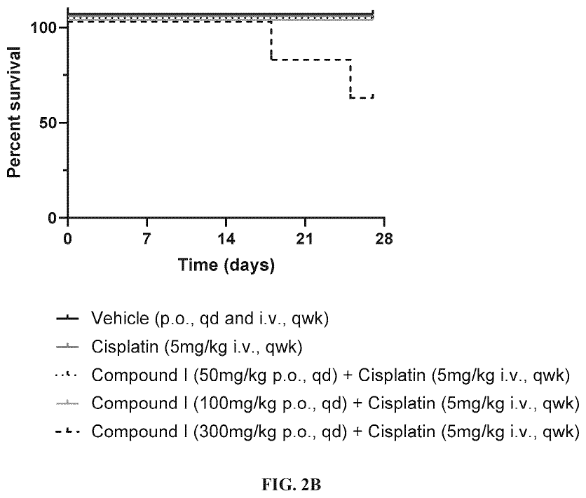

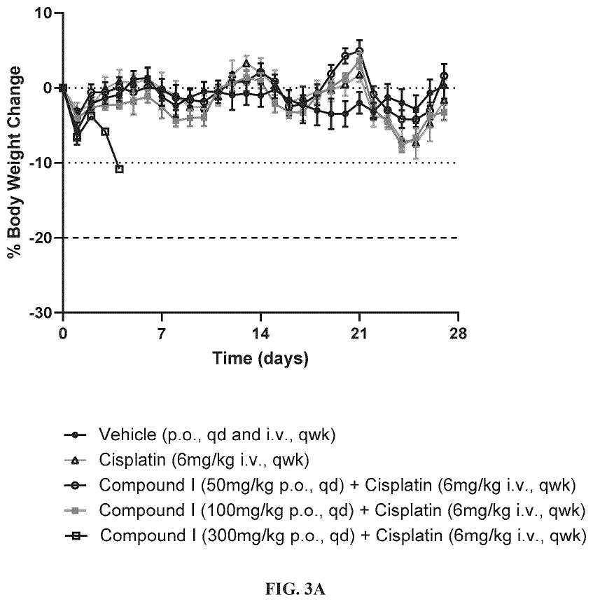

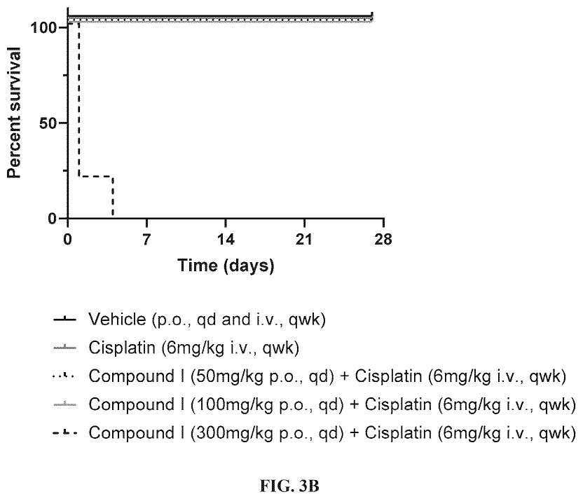

Body weights were measured daily. Tolerability was assessed by monitoring body weight and calculating body weight changes as % from body weight on day of treatment start (day 0), as shown in FIG. 1-3. If spontaneous death or body weight loss ≥20% occurred in 20% of the mice in the group, the treatment was considered not tolerated. Based on these criteria, the combination MTD was determined to be Compound 1 (100 mg/kg) and cisplatin (6 mg/kg). Being able to treat mice with 4 weekly doses of 6 mg/kg cisplatin and daily doses of 100 mg/kg Compound 1 indicates the combination is well tolerated since cisplatin is often dosed less frequently than once a week or at lower levels than 6 mg/kg in combination therapy efficacy studies with other compounds.

### Example 2: In Vitro Assays

### Colony Formation Assay

In vitro experiments were conducted using the colony formation unit (CFU) assay on various cell lines. The CFU assay involved first establishing what cell plating density enabled the development of clearly interspersed colonies on a six-well plate when left to grow for around 14 days. Once this density had been identified, cells were plated on day −1 and on day 0, the wells were treated with DMSO or 300 nM of USP1 inhibitor of Compound 1 along with increasing concentrations of temozolomide (316 nM, 1 μM, 3.16 μM, 10 μM, or 31.6 μM), cisplatin (3.16 nM, 10 nM, 31.6 nM, 100 nM, 316 nM, 1 μM, or 3.16 μM), mitomycin C (1 nM, 3.16 nM, 10 nM, 31.6 nM, 100 nM, 316 nM, 1 μM), gemcitabine (31.6 μM, 100 μM, 316 μM, 1 nM, 3.16 nM), pemetrexed (31.6 nM, 100 nM, 316 nM, 1 μM, 3.16 μM), camptothecin (316 μM, 1 nM, 3.16 nM, 10 nM, 31.6 nM, 100 nM, 316 nM), doxorubicin (10 nM, 31.6 nM, 100 nM, 316 nM, 1 μM), etoposide (31.6 nM, 100 nM, 316 nM, 1 μM, 3.16 μM), or paclitaxel (3.16 nM, 10 nM, 31.6 nM, 100 nM, 316 nM). Media was changed on day 8 containing appropriate concentrations of DMSO, USP1 inhibitor, temozolomide, cisplatin, mitomycin C, gemcitabine, pemetrexed, camptothecin, doxorubicin, etoposide, or paclitaxel. At or around day 14 when clearly interspersed colonies were visible in the DMSO-treated wells, the cells were fixed and stained using 0.1% crystal violet in 10% ethanol for 20 minutes at room temperature. The plates were imaged then the amount of crystal violet stain in each well was quantified by extracting the crystal violet into 10% acetic acid and the absorbance measured at 565 nm. The CFU results are shown in Table 1.

TABLE 1
Chemotherapy nM IC50s (synergy score)

| Lineage | Cell Line | Temozol. | Cis. | Mito. C | Gem. | Pemetrex | Campto. | Dox. | Etop. | Pacli. |
|---|---|---|---|---|---|---|---|---|---|---|
| bladder | 5637 | | 598 | | | | | | | |
| | | | (12) | | | | | | | |
| bladder | 647V | | 1777(0) | 15 | 2 | | | | | |
| | | | | (—) | (—) | | | | | |
| lung | A549 | | >316 | | | 6872 | | | | 2 |
| | | | (11) | | | (—) | | | | (—) |
| pleura | ACCMESO1 | | | 10, 13 | | 172 | | 19 | | |
| | | | | (—) | | (—) | | (—) | | |
| pancreas | ASPC1 | | | | | | 3, 3, | | | <1, 3 |
| | | | | | | | 3 (−5) | | | (—) |
| | BECKER | <1 | | | | | | | 837 | |
| | | (—) | | | | | | | (—) | |
| pancreas | CAPAN1 | | | | | | 3, <1 | | | 3, 1 |
| | | | | | | | (—) | | | (—) |
| pancreas | CAPAN2 | | | | | | 8, 11 | | | 2, <1 |
| | | | | | | | (—) | | | (—) |
| ovary | COV362 | | | | | | 274 | 108 | | 13 |
| | | | | | | | (—) | (—) | | (—) |
| nervous | GOTO | <1 | | | | | | | 11, 184 | |
| system | | (—) | | | | | | | (—) | |
| stomach | GSU | | | | | | 9, 4, | | | |
| | | | | | | | 3 (13) | | | |
| breast | HBCx8 | | 142 | | | | | | | |
| | | | (1) | | | | | | | |
| breast | HBCx9 | | 473 | | | | | | | |
| | | | (−1) | | | | | | | |
| breast | HCC1806 | | 97, 130 | 1,2, <1 | <1, <1 | | | 6, 21 | | 1, <1 |
| | | | (—) | (−3) | (—) | | | (—) | | (—) |
| breast | HCC1937 | | | 4, <1 | | | | 8, 4 | | |
| | | | | (—) | | | | (—) | | |
| colon | HCT116 | | 1259, 895 | 19 | | | | | | |
| | | | (3, 14) | (6) | | | | | | |
| colon | HCT15 | | | | | | 5 | | | |
| | | | | | | | (—) | | | |
| pancreas | HPAC | | | | | | 5, 5 | | | 3 |
| | | | | | | | (—) | | | (—) |
| bladder | HT1376 | | 734 | | | | | | | |
| | | | (10) | | | | | | | |
| nervous | KALS1 | | | 632 | | | | 52 | | |
| system | | | | (—) | | | | (—) | | |
| bladder | KU1919 | | 894 | 16 | | | | | | |
| | | | (1) | (—) | | | | | | |
| esophagus | KYSE270 | | | 15, 11 | | | | | | |
| | | | | (—) | | | | | | |
| esophagus | KYSE70 | | | 14, 12 | | | | | | |
| | | | | (—) | | | | | | |
| breast | MDAMB231 | | | 14, 14 | | | | 8, 3 | | 1, <1 |
| | | | | (—) | | | | (—) | | (—) |
| | MERO14 | | | 10, <1 | | >3000 | | 17, 14 | | |
| | | | | (—) | | (—) | | (—) | | |
| | MERO83 | | | 21, 9 | | >3000 | | 13, <1 | | |
| | | | | (—) | | (—) | | (—) | | |
| stomach | MKN7 | | | | | | 6, 2 (—) | | | |
| pleura | MSTO211H | | | 28, 9 | | 41, 50 | | 16, 8 | | |
| | | | | (—) | | (—) | | (—) | | |
| lung | NCIH1650 | | >1000 | | | 38, 2 | | | 270 | 1, <1 |
| | | | (−1) | | | (—) | | | (—) | (—) |
| lung | NCIH1915 | | | | | 185 | | | 500 | <1 |
| | | | | | | (—) | | | (—) | (—) |
| pleura | NCIH2452 | | | | | 48 | | 21 | | |
| | | | | | | (—) | | (—) | | |
| lung | NCIH596 | | 129 | 10 | | | | | | |
| | | | (0) | (−1) | | | | | | |
| ovary | OVSAHO | | | | | | 177 | 147 | | 78 |
| | | | | | | | (—) | (—) | | (—) |
| breast | SUM149PT | | >316 | 5 | 1 | | | | | |
| | | | (10) | (1) | (−1) | | | | | |
| colon | SW948 | | | | | | 10, 6 | | | |
| | | | | | | | (−3) | | | |
| esophagus | TE11 | | | 89, 11 | | | | | | |
| | | | | (—) | | | | | | |
| nervous | U251MG | <1 | | | | | | | 583 | |
| system | | (—) | | | | | | | (—) | |
| bone | U2OS | | >316 | | | | | | | |
| | | | (3) | | | | | | | |
| bladder | UBLC1 | | 308 | | | | | | | |
| | | | (−2) | | | | | | | |
| bladder | UMUC3 | | 212 | 18, 12 | | | | | | |
| | | | (1) | (—) | | | | | | |
| ovary | UWB1289 | | | | | | 18 | 76 | | 43 |
| | | | | | | | (—) | (—) | | (—) |

Temozolomide (“temozol”), cisplatin (“Cis”), mitomycin C (“Mito. C.”), gemcitabine (“gem”), pemetrexed (“pemetrex”), camptothecin (“campto”), doxorubicin (“dox.”), etoposide (“Etop”), or paclitaxel (“Pacli”).

For certain cell lines, multiple IC₅₀ values were taken, and those are separated by commas. Synergy scores are in parentheticals. For example, for the test of Compound 1 in combination with campothecin in pancreas cell line ASPC1, three separate IC₅₀ values of 3 were measured, and the synergy score was calculated to be (−5).

The results in Table 1 showed synergy was detected in cell lines treated with a combination of USP1 inhibitor and camptothecin, cisplatin, or mitomycin C. In certain cell lines, combinations of the disclosure are synergistic. In some aspects, these combinations are determined to have a Bliss score is about 6, about 7, about 8, about 9, about 10, about 11, about 12, about 13, about 14, about 15, about 16, about 17, about 18, about 19, about 20 or about 25.

### Example 3: Anti-Tumor Activity of Compound 1 Cocrystal in Combination with Cisplatin in the MDA-MB-436 Cell Line-Derived Breast Xenograft Model in NOD SCID Mice

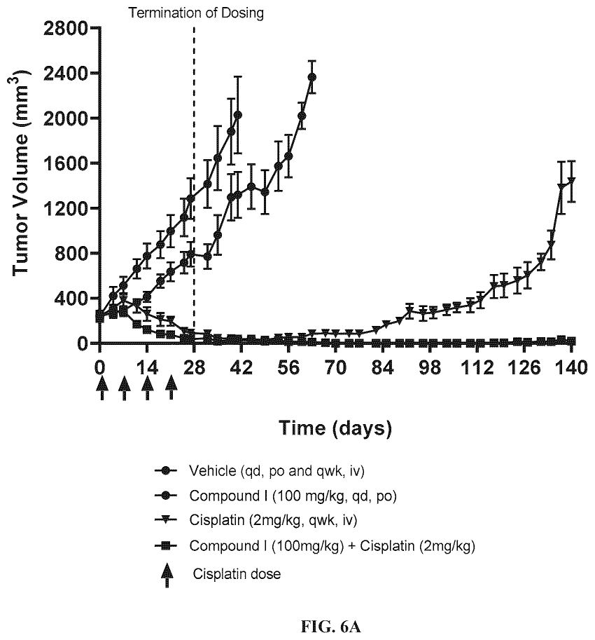

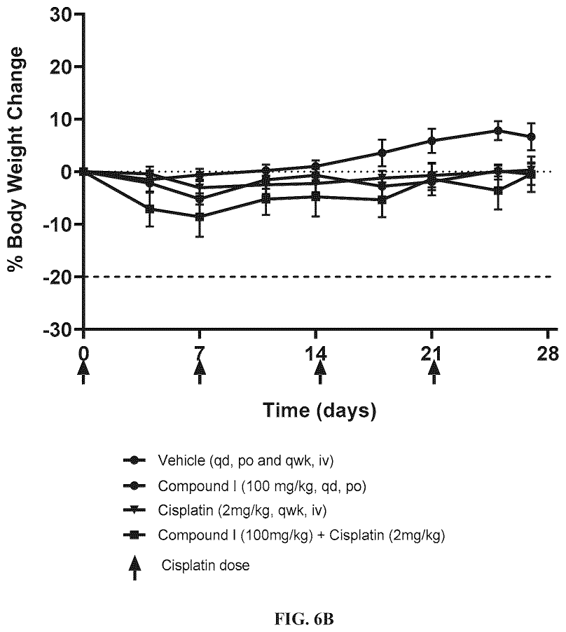

Anti-tumor activity of the USP1 inhibitor of Compound 1 cocrystal in combination with cisplatin was evaluated in mice using the MDA-MB-436 cell line-derived breast xenograft model in nonobese diabetic/severe combined immunodeficiency (NOD SCID) mice, as shown in FIG. 6A through FIG. 6B. Six to eight week old female NOD SCID mice from Beijing Anikeeper Biotech CO, Ltd were implanted subcutaneously with 1×10⁷ MDA-MB-436 cells. When tumors established to a tumor volume of approximately 240 mm³ mice were randomized into groups of 5 and were assigned into the following groups: control, cisplatin (2 mg/kg), Compound 1 (100 mg/kg) or Compound 1 (100 mg/kg) and cisplatin (2 mg/kg) combination. Cisplatin was administered intravenously via tail vein once weekly for a total of 4 doses. Compound 1 was administered via oral gavage once daily for 28 days. Body weight and tumor volume were measured twice per week. Tumor volume was calculated as mean and standard error of the mean for each treatment group.

The data in FIG. 6A show that, compared to equivalent doses of the single agent of Compound 1 or cisplatin, the combination treatment group showed a more prolonged anti-tumor activity in the MDA-MB-436 cell line-derived subcutaneous mouse model.

Tolerability of cisplatin (2 mg/kg) and Compound 1 (100 mg/kg) in the combination efficacy study, was assessed by monitoring body weight and calculating body weight changes as % from body weight on day of treatment start (day 0), as shown in FIG. 6B.

### Example 4: Anti-Tumor Activity of Compound 1 Cocrystal in Combination with Cisplatin in the HBCx-8 Patient-Derived Breast Xenograft Model in Nude Mice

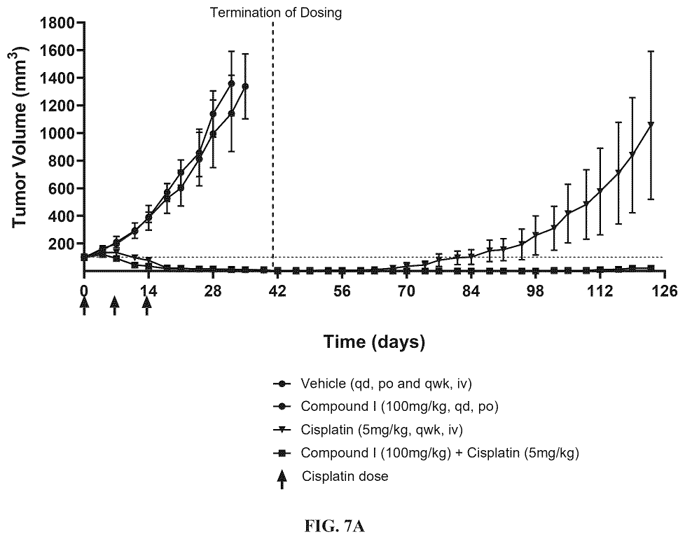

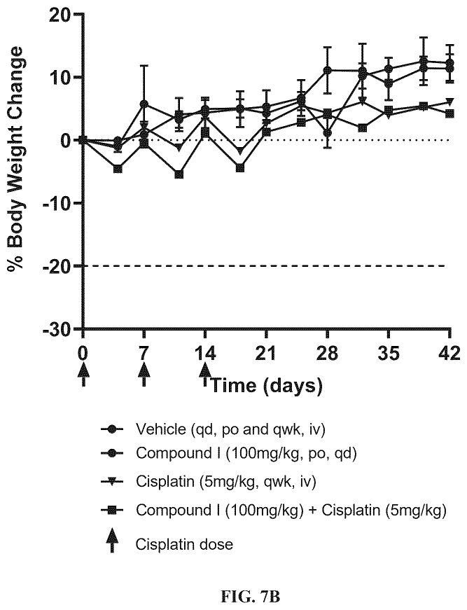

Anti-tumor activity of the USP1 inhibitor of Compound 1 cocrystal in combination with cisplatin was evaluated in mice the HBCx-8 patient-derived breast xenograft model in nude mice, as shown in FIG. 7A through FIG. 7B. Six to nine week old female Athymic nude mice from Envigo were anesthetized and a 20 mm³ tumor fragment placed subcutaneously via incision in the flank. When tumors established to a tumor volume of approximately 100 mm³ mice were randomized into groups of 3 and were assigned into the following groups: control, Compound 1 (100 mg/kg), cisplatin (5 mg/kg) or Compound 1 (100 mg/kg) and cisplatin (5 mg/kg) combination. Cisplatin was administered intravenously via tail vein once weekly for a total of 3 doses. Compound 1 was administered via oral gavage once daily for 42 days. Body weight and tumor volume were measured twice per week. Tumor volume was calculated as mean and standard error of the mean for each treatment group.

The data in FIG. 7A show that, compared to equivalent doses of the single agent of Compound 1 or cisplatin, the combination treatment group showed a more prolonged anti-tumor activity in the HBCx-8 patient-derived subcutaneous mouse model.

Tolerability of cisplatin (5 mg/kg) and Compound 1 (100 mg/kg) in the combination efficacy study, was assessed by monitoring body weight and calculating body weight changes as % from body weight on day of treatment start (day 0), as shown in FIG. 7B.

### Example 5: Anti-Tumor Activity of Compound 1 Cocrystal in Combination with Cisplatin in the HBCx-11 Patient-Derived Breast Xenograft Model in Nude Mice

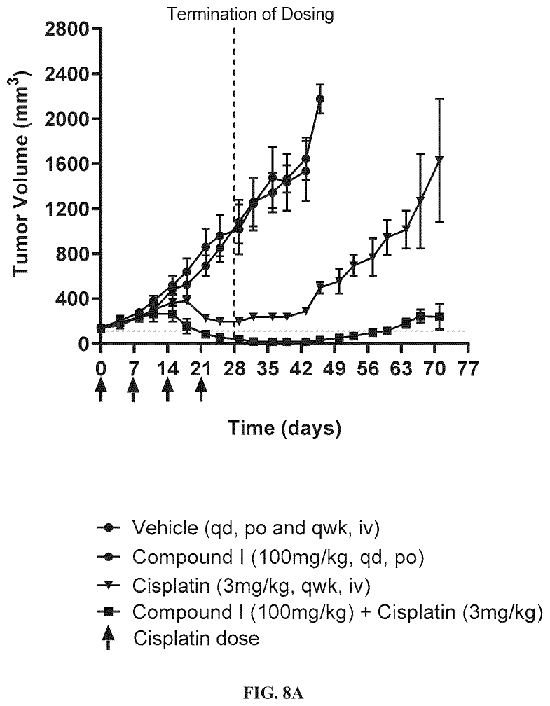

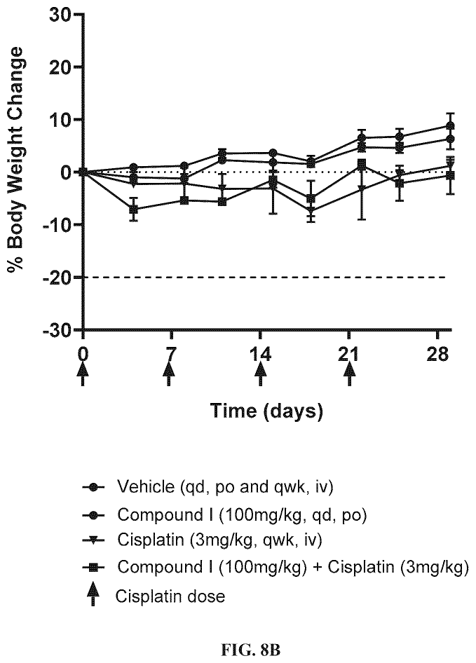

Anti-tumor activity of the USP1 inhibitor of Compound 1 cocrystal in combination with cisplatin was evaluated in mice the HBCx-11 patient-derived breast xenograft model in nude mice, as shown in FIG. 8A through FIG. 8B. Six to nine week old female Athymic nude mice from Envigo were anesthetized and a 20 mm³ tumor fragment placed subcutaneously via incision in the flank. When tumors established to a tumor volume of between 75 and 196 mm³ mice were randomized into groups of 3 and were assigned into the following groups: control, Compound 1 (100 mg/kg), cisplatin (3 mg/kg) or Compound 1 (100 mg/kg) and cisplatin (3 mg/kg) combination. Cisplatin was administered intravenously via tail vein once weekly for a total of 4 doses. Compound 1 was administered via oral gavage once daily for 28 days. Body weight and tumor volume were measured twice per week. Tumor volume was calculated as mean and standard error of the mean for each treatment group.

The data in FIG. 8A show that, compared to equivalent doses of the single agent of Compound 1 or cisplatin, the combination treatment group showed a superior and more prolonged anti-tumor activity in the HBCx-11 patient-derived subcutaneous mouse model.

Tolerability of cisplatin (3 mg/kg) and Compound 1 (100 mg/kg) in the combination efficacy study, was assessed by monitoring body weight and calculating body weight changes as % from body weight on day of treatment start (day 0), as shown in FIG. 8B.

All patents and publications cited herein are fully incorporated by reference herein in their entirety.

1. A method of treating cancer in a subject, comprising administering to the subject (i) a chemotherapy agent and (ii) a ubiquitin-specific-processing protease 1 (USP1) inhibitor having Formula I: 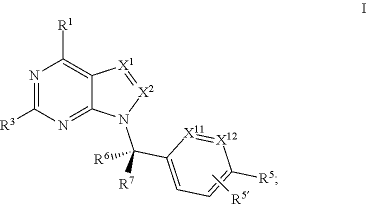
or a pharmaceutically acceptable salt or solvate thereof, wherein:
each of X¹ and X² is independently selected from N and CR²;
each of R¹ and R² is independently selected from hydrogen, halo, cyano, optionally substituted alkyl, optionally substituted alkenyl, and optionally substituted alkynyl;
R³ is an optionally substituted phenyl, optionally substituted pyridyl, optionally substituted pyrimidinyl, optionally substituted pyrazinyl, optionally substituted pyridazinyl, or optionally substituted pyrazolyl;
each of X¹¹ and X¹² is independently selected from N and CH;
R^{5′} is selected from hydrogen, optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted —O-heterocyclo, optionally substituted —O-heterocyclo-(C₁-C₂) alkyl;
R⁵ is selected from optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O— ((C₃-C₈) cycloalkyl)-(C₁-C₂) alkyl, optionally substituted —O-heterocyclo, optionally substituted —O-heterocyclo-(C₁-C₂) alkyl; or one of R⁵ and one of R^{5′} on adjacent atoms are taken together with the atoms to which they are attached to form an optionally substituted (C₆-C₁₄) aryl ring; or one of R⁵ and one of R^{5′} on adjacent atoms are taken together with the atoms to which they are attached to form an optionally substituted heteroaryl ring; or one of R⁵ and one of R^{5′} on adjacent atoms are taken together with the atoms to which they are attached to form an optionally substituted (C₃-C₈) cycloalkyl ring; or one of R⁵ and one of R^{5′} on adjacent atoms are taken together with the atoms to which they are attached to form an optionally substituted heterocycloalkyl ring; or one of R⁵ and one of R^{5′} on adjacent atoms on the same atom to which they are attached are taken together to form an optionally substituted spirocycloalkyl ring; or one of R⁵ and one of R^{5′} on adjacent atoms on the same atom to which they are attached are taken together to form an optionally substituted spiroheterocycloalkyl ring;
each of R⁶ and R⁷ is independently selected from hydrogen, halo, cyano, optionally substituted alkyl, optionally substituted alkenyl, and optionally substituted alkynyl;
R²³ is selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, optionally substituted heterocyclo, optionally substituted heteroaryl, optionally substituted aryl, and optionally substituted cycloalkyl;
R^{31a} and R^{31b} are each independently selected from the group consisting of hydrogen, optionally substituted alkyl, optionally substituted alkenyl, optionally substituted alkynyl, hydroxyalkyl, (amino)alkyl, (alkylamino)alkyl, (dialkylamino)alkyl, alkoxyalkyl, cycloalkyl, (cycloalkyl)alkyl, (heterocyclo)alkyl, aralkyl, and (heteroaryl)alkyl; and
each of R²⁴, R²⁵, R²⁷, R^{32a}, and R^{32b} is independently selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, alkoxyalkyl, optionally substituted heterocyclo, optionally substituted heteroaryl, optionally substituted aryl, and optionally substituted cycloalkyl.

2. A method of delaying, reducing, and/or preventing rebounding of a tumor in a subject comprising administering to the subject (i) a chemotherapy agent and (ii) a ubiquitin-specific-processing protease 1 (USP1) inhibitor having Formula I: 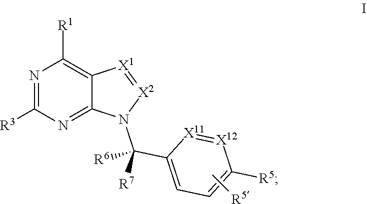
or a pharmaceutically acceptable salt or solvate thereof, wherein:
each of X¹ and X² is independently selected from N and CR²;
each of R¹ and R² is independently selected from hydrogen, halo, cyano, optionally substituted alkyl, optionally substituted alkenyl, and optionally substituted alkynyl;
R³ is an optionally substituted phenyl, optionally substituted pyridyl, optionally substituted pyrimidinyl, optionally substituted pyrazinyl, optionally substituted pyridazinyl, or optionally substituted pyrazolyl;
each of X¹¹ and X¹² is independently selected from N and CH;
R^{5′} is selected from hydrogen, optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted —O-heterocyclo, optionally substituted —O-heterocyclo-(C₁-C₂) alkyl;
R⁵ is selected from optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted —O-heterocyclo, optionally substituted —O-heterocyclo-(C₁-C₂) alkyl; or one of R⁵ and one of R^{5′} on adjacent atoms are taken together with the atoms to which they are attached to form an optionally substituted (C₆-C₁₄) aryl ring; or one of R⁵ and one of R^{5′} on adjacent atoms are taken together with the atoms to which they are attached to form an optionally substituted heteroaryl ring; or one of R⁵ and one of R^{5′} on adjacent atoms are taken together with the atoms to which they are attached to form an optionally substituted (C₃-C₈) cycloalkyl ring; or one of R⁵ and one of R^{5′} on adjacent atoms are taken together with the atoms to which they are attached to form an optionally substituted heterocycloalkyl ring; or one of R⁵ and one of R^{5′} on adjacent atoms on the same atom to which they are attached are taken together to form an optionally substituted spirocycloalkyl ring; or one of R⁵ and one of R^{5′} on adjacent atoms on the same atom to which they are attached are taken together to form an optionally substituted spiroheterocycloalkyl ring;
each of R⁶ and R⁷ is independently selected from hydrogen, halo, cyano, optionally substituted alkyl, optionally substituted alkenyl, and optionally substituted alkynyl;
R²³ is selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, optionally substituted heterocyclo, optionally substituted heteroaryl, optionally substituted aryl, and optionally substituted cycloalkyl;
R^{31a} and R^{31b} are each independently selected from the group consisting of hydrogen, optionally substituted alkyl, optionally substituted alkenyl, optionally substituted alkynyl, hydroxyalkyl, (amino)alkyl, (alkylamino)alkyl, (dialkylamino)alkyl, alkoxyalkyl, cycloalkyl, (cycloalkyl)alkyl, (heterocyclo)alkyl, aralkyl, and (heteroaryl)alkyl; and
each of R²⁴, R²⁵, R²⁷, R^{32a}, and R^{32b} is independently selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, alkoxyalkyl, optionally substituted heterocyclo, optionally substituted heteroaryl, optionally substituted aryl, and optionally substituted cycloalkyl.

3. The method of claim 1, wherein the chemotherapy agent is a platin, a monofunctional alkylator, a bifunctional alkylator, an antimetabolite, a topoisomerase inhibitor, or a combination thereof.

4. The method of claim 3, wherein the chemotherapy agent is a platin selected from the group consisting of cisplatin, carboplatin, oxaliplatin, satraplatin, picoplatin, nedaplatin, and triplatin; optionally, wherein the platin is cisplatin or carboplatin.

5. The method of claim 3, wherein the chemotherapy agent is a monofunctional alkylator selected from the group consisting of an alkylsulfonate, a nitrosourea, and temozolomide; or a bifunctional alkylator selected from the group consisting of nitrogen mustard and mitomycin C.

6. The method of claim 3, wherein the chemotherapy agent is an antimetabolite selected from the group consisting of gemcitabine, 5-fluorouracil, a thiopurine, and a folate analog.

7. The method of claim 3, wherein the chemotherapy agent is a topoisomerase inhibitor selected from the group consisting of a campothecin, an anthracycline, and an etoposide.

8. The method of any one of claim 1, wherein the compound of Formula I is provided as a cocrystal formed between the USP1 inhibitor of Formula I and a pharmaceutically acceptable acid.

9. The method of any one of claim 1, wherein the USP1 inhibitor has one of the following structures: 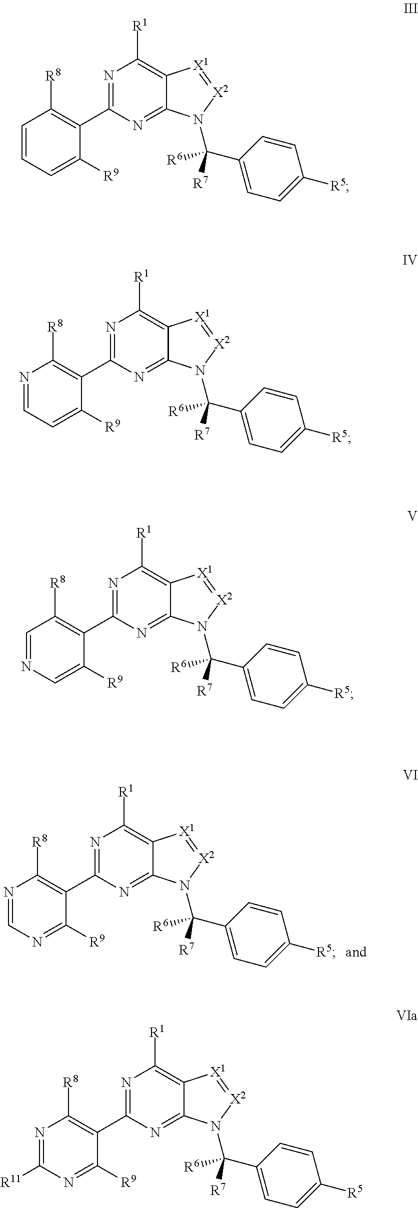
or a pharmaceutically acceptable salt or solvate thereof, wherein
each of X¹ and X² is independently selected from N and CR²;
R¹ is selected from hydrogen, halo, cyano, optionally substituted alkyl, optionally substituted alkenyl, and optionally substituted alkynyl;
R⁵ is: 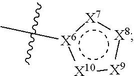
X⁶ is selected from NR¹³ and CR⁸;
X⁷ is selected from NR¹⁴ and CR¹⁹;
X⁸ is selected from NR¹⁵ and CR²⁰;
X⁹ is selected from NR¹⁶ and CR²¹;
X¹⁰ is selected from NR¹⁷ and CR²²;
each of R¹³, R¹⁴, R¹⁵, R¹⁶, and R¹⁷ is absent, or independently selected from hydrogen, halo, methyl, ethyl, isopropyl, cyclopropyl, methoxy, triazolyl, cyano, optionally substituted alkyl, amino, alkylamino, dialkylamino, difluoromethyl, trifluoromethyl, methylsulfonyl, and methylazetidinyl; and
each of R¹⁸, R¹⁹, R²⁰, R²¹, and R²² is independently selected from hydrogen, halo, methyl, ethyl, isopropyl, cyclopropyl, methoxy, triazolyl, cyano, optionally substituted alkyl, amino, alkylamino, dialkylamino, difluoromethyl, trifluoromethyl, methylsulfonyl, and methylazetidinyl;
each of R⁶ and R⁷ is independently selected from hydrogen, halo, cyano, optionally substituted alkyl, optionally substituted alkenyl, and optionally substituted alkynyl; and
each of R⁸, R⁹ and R¹¹ is independently selected from the group consisting of hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, or (heteroaryl)alkyl.

10. The method of claim 1, wherein the USP1 inhibitor is a pharmaceutically acceptable salt, hydrate, solvate, amorphous solid, polymorph, or cocrystal thereof.

11. The method of claim 1, wherein the USP1 inhibitor is a compound selected from the group consisting of:
(a) Compound 1: 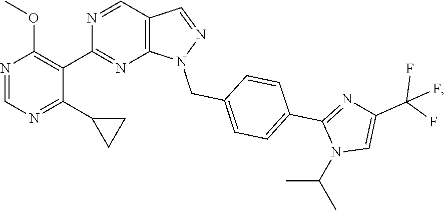 [SMILES: COc1ncnc(C2CC2)c1-c1ncc2cnn(Cc3ccc(-c4nc(C(F)(F)F)cn4C(C)C)cc3)c2n1]
(b) Compound 2: 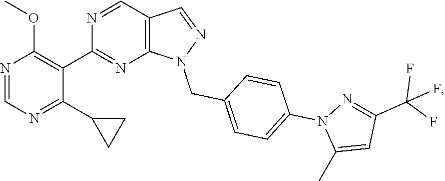 [SMILES: COc1ncnc(C2CC2)c1-c1ncc2cnn(Cc3ccc(-n4nc(C(F)(F)F)cc4C)cc3)c2n1]
(c) Compound 3: 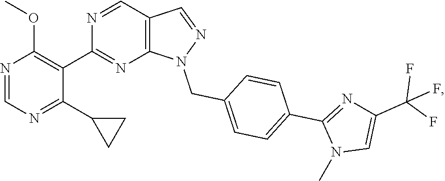 [SMILES: COc1ncnc(C2CC2)c1-c1ncc2cnn(Cc3ccc(-c4nc(C(F)(F)F)cn4C)cc3)c2n1]
and a pharmaceutically acceptable salt, hydrate, solvate, amorphous solid, or polymorph thereof; or
a pharmaceutically acceptable salt thereof; or
a hydrate thereof; or
a solvate thereof; or
an amorphous solid thereof; or
a polymorph thereof, or
a cocrystal thereof.

12. The method of claim 11, wherein the USP1 inhibitor is Compound 1, or a pharmaceutically acceptable salt, hydrate, solvate, amorphous solid, or polymorph thereof; or
wherein the USP1 inhibitor is Compound 1, or a cocrystal thereof, or
wherein the USP1 inhibitor is Compound 2, or a pharmaceutically acceptable salt, hydrate, solvate, amorphous solid, or polymorph thereof, or
wherein the USP1 inhibitor is Compound 2, or a cocrystal thereof, or
wherein the USP1 inhibitor is Compound 3, or a pharmaceutically acceptable salt, hydrate, solvate, amorphous solid, or polymorph thereof, or
wherein the USP1 inhibitor is Compound 3, or a cocrystal thereof.

13. The method of claim 1, wherein the USP1 inhibitor is provided as a cocrystal formed between 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine and a pharmaceutically acceptable acid.

14. The method of claim 13, wherein the pharmaceutically acceptable acid is selected from the group consisting of gentisic acid, benzoic acid, salicylic acid, and gallic acid; optionally, wherein the pharmaceutically acceptable acid is gentisic acid.

15. (canceled)

16. The method of claim 1, wherein the cancer cells of said subject are HRR positive.

17-20. (canceled)

21. The method of claim 1, wherein the chemotherapy agent and the USP1 inhibitor are administered in a therapeutically effective amount sufficient to produce one or more therapeutic effects selected from the group consisting of (i) reduction in tumor size, (ii) increase in tumor regression rate, and (iii) reduction or inhibition of tumor growth; or
wherein the chemotherapy agent and the USP1 inhibitor are administered in an amount effective to reduce unacceptable toxicity and/or unacceptable adverse reactions of a chemotherapy agent administered as a monotherapy.

22. (canceled)

23. The method of any claim 1, wherein the USP1 inhibitor, gemcitabine and carboplatin are administered; optionally wherein the subject has ovarian cancer, triple negative breast cancer, or non-small cell lung cancer.

24. The method of claim 1, wherein the USP1 inhibitor, leucovorin calcium, 5-fluorouracil, and oxaliplatin are administered; optionally, wherein the subject has colorectal cancer, pancreatic cancer, esophageal cancer, or gastric cancer.

25. The method of claim 1, wherein the cancer or tumor was previously treated with a platinum therapy, optionally wherein the cancer or tumor is platinum resistant or platinum refractory; optionally wherein the platinum therapy is selected from the group consisting of cisplatin, carboplatin, oxaliplatin, and combinations thereof.

26. A combination comprising (i) a chemotherapy agent and (ii) a ubiquitin-specific-processing protease 1 (USP1) inhibitor, wherein USP1 inhibitor is a compound having Formula I: 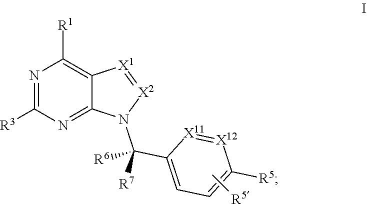
or a pharmaceutically acceptable salt or solvate thereof, wherein:
each of X¹ and X² is independently selected from N and CR²;
each of R¹ and R² is independently selected from hydrogen, halo, cyano, optionally substituted alkyl, optionally substituted alkenyl, and optionally substituted alkynyl;
R³ is an optionally substituted phenyl, optionally substituted pyridyl, optionally substituted pyrimidinyl, optionally substituted pyrazinyl, optionally substituted pyridazinyl, or optionally substituted pyrazolyl;
each of X¹¹ and X¹² is independently selected from N and CH;
R^{5′} is selected from hydrogen, optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted —O-heterocyclo, optionally substituted —O-heterocyclo-(C₁-C₂) alkyl;
R⁵ is selected from optionally substituted (C₁-C₆) alkyl, optionally substituted (C₂-C₆) alkenyl, optionally substituted (C₂-C₆) alkynyl, optionally substituted (C₁-C₆) alkoxy, (C₁-C₆) haloalkyl, (C₁-C₆) haloalkoxy, (C₁-C₆) hydroxyalkyl, cyano, halo, sulfonamido, —C(═O)R²³, —C(═O)OR²⁴, —NR^{32a}R^{32b}, —NR^{31a}C(═O)R²⁵, —NR^{31a}C(═O)NR^{31a}R^{31b}, —C(═O)NR^{31a}R^{31b}, —S(O)₂R²⁷, —NR^{31a}SO₂R²⁷, optionally substituted (C₆-C₁₄) aryl, optionally substituted (C₆-C₁₄) ar-(C₁-C₂) alkyl, optionally substituted heteroaryl, optionally substituted heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted —O—(C₆-C₁₄) aryl, optionally substituted —O—(C₆-C₁₄) ar-(C₁-C₂) alkyl, optionally substituted —O-heteroaryl, optionally substituted —O-heteroar-(C₁-C₂) alkyl, optionally substituted —O—(C₃-C₈) cycloalkyl, optionally substituted —O—((C₃-C₈) cycloalkyl)-(C₁-C₂) alkyl, optionally substituted —O-heterocyclo, optionally substituted —O-heterocyclo-(C₁-C₂) alkyl; or
one of R⁵ and one of R^{5′} on adjacent atoms are taken together with the atoms to which they are attached to form an optionally substituted (C₆-C₁₄) aryl ring; or one of R⁵ and one of R^{5′} on adjacent atoms are taken together with the atoms to which they are attached to form an optionally substituted heteroaryl ring; or one of R⁵ and one of R^{5′} on adjacent atoms are taken together with the atoms to which they are attached to form an optionally substituted (C₃-C₈) cycloalkyl ring; or one of R⁵ and one of R^{5′} on adjacent atoms are taken together with the atoms to which they are attached to form an optionally substituted heterocycloalkyl ring; or one of R⁵ and one of R^{5′} on adjacent atoms on the same atom to which they are attached are taken together to form an optionally substituted spirocycloalkyl ring; or one of R⁵ and one of R^{5′} on adjacent atoms on the same atom to which they are attached are taken together to form an optionally substituted spiroheterocycloalkyl ring;
each of R⁶ and R⁷ is independently selected from hydrogen, halo, cyano, optionally substituted alkyl, optionally substituted alkenyl, and optionally substituted alkynyl;
R²³ is selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, optionally substituted heterocyclo, optionally substituted heteroaryl, optionally substituted aryl, and optionally substituted cycloalkyl;
R^{31a} and R^{31b} are each independently selected from the group consisting of hydrogen, optionally substituted alkyl, optionally substituted alkenyl, optionally substituted alkynyl, hydroxyalkyl, (amino)alkyl, (alkylamino)alkyl, (dialkylamino)alkyl, alkoxyalkyl, cycloalkyl, (cycloalkyl)alkyl, (heterocyclo)alkyl, aralkyl, and (heteroaryl)alkyl; and
each of R²⁴, R²⁵, R²⁷, R^{32a}, and R^{32b} is independently selected from the group consisting of hydrogen, optionally substituted alkyl, optionally substituted alkenyl, optionally substituted alkynyl, amino, alkylamino, dialkylamino, cycloalkylamino, hydroxyalkyl, (amino)alkyl, (alkylamino)alkyl, (dialkylamino)alkyl, (cycloalkylamino)alkyl, (cycloalkyl)alkyl, aralkyl, (heterocyclo)alkyl, (heteroaryl)alkyl, (amino)(hydroxy)alkyl, (aralkylamino)alkyl, alkoxyalkyl, optionally substituted heterocyclo, optionally substituted heteroaryl, optionally substituted aryl, and optionally substituted cycloalkyl.

27-42. (canceled)


SEQUENCE LISTING (1 sequence)

SEQ ID NO: 1 - protein, 785 aa, Homo sapiens
  source 1..785: note = human USP1 protein
MPGVIPSESNGLSRGSPSKKNRLSLKFFQKKETKRALDFTDSQENEEKASEYRASEIDQVVPAAQSSPINCEKRENLLPFVGLNNLGNTCYLNSILQVLYFCPGFKSGVKHLFNIISRKKEALKDEANQKDKGNCKEDSLASYELICSLQSLIISVEQLQASFLLNPEKYTDELATQPRRLLNTLRELNPMYEGYLQHDAQEVLQCILGNIQETCQLLKKEEVKNVAELPTKVEEIPHPKEEMNGINSIEMDSMRHSEDFKEKLPKGNGKRKSDTEFGNMKKKVKLSKEHQSLEENQRQTRSKRKATSDTLESPPKIIPKYISENESPRPSQKKSRVKINWLKSATKQPSILSKFCSLGKITTNQGVKGQSKENECDPEEDLGKCESDNTTNGCGLESPGNTVTPVNVNEVKPINKGEEQIGFELVEKLFQGQLVLRTRCLECESLTERREDFQDISVPVQEDELSKVEESSEISPEPKTEMKTLRWAISQFASVERIVGEDKYFCENCHHYTEAERSLLFDKMPEVITIHLKCFAASGLEFDCYGGGLSKINTPLLTPLKLSLEEWSTKPTNDSYGLFAVVMHSGITISSGHYTASVKVTDLNSLELDKGNFVVDQMCEIGKPEPLNEEEARGVVENYNDEEVSIRVGGNTQPSKVLNKKNVEAIGLLGGQKSKADYELYNKASNPDKVASTAFAENRNSETSDTTGTHESDRNKESSDQTGINISGFENKISYVVQSLKEYEGKWLLFDDSEVKVTEEKDFLNSLSPSTSPTSTPYLLFYKKL